SEQUENCE LISTING (4 sequences)

SEQ ID NO: 1 - DNA, 40 bp, Artificial Sequence
  oligonucleotide primer
gcaggaaacagctatgaccatacagtcagcggccgccacc

SEQ ID NO: 2 - DNA, 39 bp, Artificial Sequence
  oligonucleotide primer
acggccagtcgcgtggagtgttatgtcatgcggccgcta

SEQ ID NO: 3 - DNA, 10625 bp, Homo sapiens
  CDS (544)..(6960)
cttattttgaaaacatttacatagtgattagttaacccaacagaccaatcctgggaagacagccagagcctgcagcaccttagtaacagaaaaactgataattaggagaagagacctgtccaagaccaggaacctggaccaaaattgtgccatgttgctttactttaatgagtggccccagtaaaaactgagctgtatggcagagctgttcacatttatcttctgtgtccacccagttctgctgaaacccctggcaagatcgtggccctgttgtagcttgtcatgttttgaacagctgtctatggaaagaaagcaaacacaacctagagcaacattgatttgttttagaaagctcttttattttcagttctggctgtgttcaacatcttagcttacgtttttcatgttgtaatgatctgccgtatggacgatcacctctaagttagagagttctgtaatttggcttggattaaagatgcttggttagtgaaagctgctgctttttttatagtcaaaggactggttctgagagccttgttgcagatggctgaggtcaccgtcccaagggtgtatgtcgtgtttggcatccattgcatcatggcgaaggcatcttcagatgtgcaggtttcaggctttcatcggaaaatccagcacgttaaaaatgaactttgccacatgttgagcttggaggaggtggccccagtgctgcagcagacattacttcaggacaacctcttgggcagggtacattttgaccaatttaaagaagcattaatactcatcttgtccagaactctgtcaaatgaagaacactttcaagaaccagactgctcactagaagctcagcccaaatatgttagaggtgggaagcgttacggacgaaggtccttgcccgagttccaagagtccgtggaggagtttcctgaagtgacggtgattgagccactggatgaagaagcgcggccttcacacatcccagccggtgactgcagtgagcactggaagacgcaacgcagtgaggagtatgaagcggaaggccagttaaggttttggaacccagatgacttgaatgcttcacagagtggatcttcccctccccaagactggatagaagagaaactgcaagaagtttgtgaagatttggggatcacccgtgatggtcacctgaaccggaagaagctggtctccatctgtgagcagtatggtttacagaatgtggatggagagatgctcgaggaagtattccataatcttgatcctgacggtacaatgagtgtagaagattttttctatggtttgtttaaaaatggaaaatctcttacaccatcagcatctactccatatagacaactaaaaaggcacctttccatgcagtctttcgatgagagtggacgacgtaccacaacctcatcagcaatgacaagtaccattggctttcgggtcttctcctgcctggatgatgggatgggccatgcatctgtggagagaatactggacacctggcaggaagagggcattgagaacagccaggagatcctgaaggccttggatttcagcctcgatggaaacatcaatttgacagaattaacactggcccttgaaaatgaacttttggttaccaagaacagcattcaccaggcggctctggccagctttaaggctgaaatccggcatttgttggaacgagttgatcaggtggtcagagaaaaagagaagctacggtcagatctggacaaggccgagaagctcaagtctttaatggcctcggaggtggatgatcaccatgcggccatagagcggcggaatgagtacaacctcaggaaactggatggagagtacaaggagcgaatagcagccttaaaaaatgaactccgaaaagagagagagcagatcctgcagcaggcaggcaagcagcgtttagaacttgaacaggaaattgaaaaggcaaaaacagaagagaactatatccgggaccgccttgccctctctttaaaggaaaacagtcgtctggaaaatgagcttctagaaaatgcagagaagttggcagaatatgagaatctgacaaacaaacttcagagaaatttggaaaatgtgttagcagaaaagtttggtgacctcgatcctagcagtgctgagttcttcctgcaagaagagagactgacacagatgagaaatgaatatgagcggcagtgcagggtactacaagaccaagtagatgaactccagtctgagctggaagaatatcgtgcacaaggcagagtgctcaggcttccgttgaagaactcaccgtcagaagaagttgaggctaacagcggtggcattgagcccgaacacgggctcggttctgaagaatgcaatccattgaatatgagcattgaggcagagctggtcattgaacagatgaaagaacaacatcacagggacatatgttgcctcagactggagctcgaagataaagtgcgccattatgaaaagcagctggacgaaaccgtggtcagctgcaagaaggcacaggagaacatgaagcaaaggcatgagaacgaaacgcgcaccttagaaaaacaaataagtgaccttaaaaatgaaattgctgaacttcaggggcaagcagcagtgctcaaggaggcacatcatgaggccacttgcaggcatgaggaggagaaaaaacaactgcaagtgaagcttgaggaggaaaagactcacctgcaggagaagctgaggctgcaacatgagatggagctcaaggctagactgacacaggctcaagcaagctttgagcgggagagggaaggccttcagagtagcgcctggacagaagagaaggtgagaggcttgactcaggaactagagcagtttcaccaggagcagctgacaagcctggtggagaaacacactcttgagaaagaggagttaagaaaagagctcttggaaaagcaccaaagggagcttcaggagggaagggaaaaaatggaaacagagtgtaatagaagaacctctcaaatagaagcccagtttcagtctgattgtcagaaagtcactgagaggtgtgaaagcgctctgcaaagcctggaggggcgctaccgccaagagctgaaggacctccaggaacagcagcgtgaggagaaatcccagtgggaatttgagaaggacgagctcacccaggagtgtgcggaagcccaggagctgctgaaagagactcttaagagagagaaaacaacttctctggtcctgacccaggagagagagatgctggagaaaacatacaaagaacatttgaacagcatggtcgtcgagagacagcagctactccaagacctggaagacctaagaaatgtatctgaaacccagcaaagcctgctgtctgaccagatacttgagctgaagagcagtcacaaaagggaactgagggagcgtgaggaggtcctgtgccaggcaggggcttcggagcagctggccagccagcggctggaaagactagaaatggaacatgaccaggaaaggcaggaaatgatgtccaagcttctagccatggagaacattcacaaagcgacctgtgagacagcagatcgagaaagagccgagatgagcacagaaatctccagacttcagagtaaaataaaggaaatgcagcaggcaacatctcctctctcaatgcttcagagtggttgccaggtgataggagaggaggaggtggaaggagatggagccctgtccctgcttcagcaaggggagcagctgttggaagaaaatggggacgtcctcttaagcctgcagagagctcatgaacaggcagtgaaggaaaatgtgaaaatggctactgaaatttctagattgcaacagaggctacaaaagttagagccagggttagtaatgtcttcttgtttggatgagccagctactgagttttttggaaatactgcggaacaaacagagcagtttttacagcaaaaccgaacgaagcaagtagaaggtgtgaccaggcggcatgtcctaagtgacctggaagatgatgaggtccgggacctgggaagtacagggacgagctctgttcagagacaggaagtcaaaatagaggagtctgaagcttcagtagagggtttttctgagcttgaaaacagtgaagagaccaggactgaatcctgggagctgaagaatcagattagtcagcttcaggaacagctaatgatgttatgtgcggactgtgatcgagcttctgaaaagaaacaggacctactttttgatgtttctgtgctaaaaaagaaactgaagatgcttgagagaatccctgaggcttctcccaaatataagctgttgtatgaagatgtgagccgagaaaatgactgccttcaggaagagctgagaatgatggagacacgctacgatgaggcactagaaaataacaaagaactcactgcagaggttttcaggttgcaggatgagctgaagaaaatggaggaagtcactgaaacattcctcagcctggaaaagagttacgatgaggtcaaaatagaaaatgaggggctgaatgttctggttttgagacttcaaggcaagattgagaagcttcaggaaagcgtggtccagcggtgtgactgctgcttatgggaagccagtttagagaacctggaaatcgaacctgatggaaatatactccagctcaatcagacactggaagagtgtgtgcccagggttaggagtgtacatcatgtcatagaggaatgtaagcaagaaaaccagtaccttgaggggaacacacagctcttggaaaaagtaaaagcacatgaaattgcctggttacatggaacaattcagacacatcaagaaaggccaagagtacagaatcaagttatactggaggaaaacactactctcctaggctttcaagacaaacattttcagcatcaggccaccatagcagagttagaactggagaaaacaaagttacaggagctgactaggaagttgaaggagagagtcactattttagttaagcaaaaagatgtactttctcacggagaaaaggaggaagagctgaaggcaatgatgcatgacttgcagatcacgtgcagtgagatgcagcaaaaagttgaacttctgagatatgaatctgaaaagcttcaacaggaaaattctattttgagaaatgaaattactactttaaatgaagaagatagcatttctaacctgaaattagggacattaaatggatctcaggaagaaatgtggcaaaaaacggaaactgtaaaacaagaaaatgctgcagttcagaagatggttgaaaatttaaagaaacagatttcagaattaaaaatcaaaaaccaacaattggatttggaaaatacagaacttagccaaaagaactctcaaaaccaggaaaaactgcaagaacttaatcaacgtctaacagaaatgctatgccagaaggaaaaagagccaggaaacagtgcattggaggaacgggaacaagagaagtttaatctgaaagaagaactggaacgttgtaaagtgcagtcctccactttagtgtcttctctggaggcggagctctctgaagttaaaatacagacccatattgtgcaacaggaaaaccaccttctcaaagatgaactggagaaaatgaaacagctgcacagatgtcccgatctctctgacttccagcaaaaaatctctagtgttctaagctacaacgaaaaactgctgaaagaaaaggaagctctgagtgaggaattaaatagctgtgtcgataagttggcaaaatcaagtcttttagagcatagaattgcgacgatgaagcaggaacagaaatcctgggaacatcagagtgcgagcttaaagtcacagctggtggcttctcaggaaaaggttcagaatttagaagacaccgtgcagaatgtaaacctgcaaatgtcccggatgaaatctgacctacgagtgactcagcaggaaaaggaggctttaaaacaagaagtgatgtctttacataagcaacttcagaatgctggtggcaagagctgggccccagagatagctactcatccatcagggctccataaccagcagaaaaggctgtcctgggacaagttggatcatctgatgaatgaggaacagcagctgctttggcaagagaatgagaggctccagaccatggtacagaacaccaaagccgaactcacgcactcccgggagaaggtccgtcaattggaatccaatcttcttcccaagcaccaaaaacatctaaacccatcaggtaccatgaatcccacagagcaagaaaaattgagcttaaagagagagtgtgatcagtttcagaaagaacaatctcctgctaacaggaaggtcagtcagatgaattcccttgaacaagaattagaaacaattcatttggaaaatgaaggcctgaaaaagaaacaagtaaaactggatgagcagctcatggagatgcagcacctgaggtccactgcgacgcctagcccgtcccctcatgcttgggatttgcagctgctccagcagcaagcctgtccgatggtgcccagggagcagtttctgcagcttcaacgccagctgctgcaggcagaaaggataaaccagcacctgcaggaggaacttgaaaacaggacctccgaaaccaacacaccacagggaaaccaggaacaactggtaactgtcatggaggaacgaatgatagaagttgaacagaaactgaaactagtgaaaaggcttcttcaagagaaagtgaatcagctcaaagaacaactctgcaagaacactaaggcagacgcaatggtgaaggacttgtatgttgaaaatgcccagttgttgaaagctctggaagtgactgaacagcgacagaaaacagcagagaagaaaaattacctcctggaggagaagattgccagcctcagtaatatagttaggaatctgacaccagcgccattgacttctacacctcctttgaggtcatagccaaaccaaagggtacactcatatttgtgcactttactgaaatagatgaacatttcagtaggttctcaacttaaaattaagcctaacctaaaactgccagcaacacaactggagtttccatttatcataattagtttttctaaatagacccttatgggagtttgaaaataaatactcacatatttcactacttaaattattcccaagatttgaatttattttaaaattttaatagccaccaagaatgtggacatatgaaaattcaagaacctaaaaaataccagttttgaatgagtttttgtggttttggttttttaattattacaaatctatgtgtaaaatctagatatttgaagtttgagatctgatgagaatggttgttataaactttattttaaaaccaaatttaggtgttcttacatatttaaatactggaaagtcattataatagttttggttctttgaattggtagacaattagtagagtataattggttaggaggcagggcttattaagtggttattaaccgctgacatcagacaaacccaaatctgtagaattctaacctcctaacacctgtgacagtattaccactcttcttgtattatagatttagaactgatttactcaattgcactcttaactaatgttaaaagcttacttgctttaaacagccttttcttctttctcttaaaagtttcatttggggagctggtcttctaagaaacggataaagccacataattaaagcagttgaactagagggaaagcactgaacaaaccactttggagtaaatagctactcttagaaaagagggataagcagaccatgtaggttttctgtctctcaaatcttagagttcataaatttacttgaggttgcctcaagaactcagggaacaatactgtaaactgtcttcctgaactactgtagggcctctctaagaatttgaaatgtataaaccatgtgacctcatttatttgtcttatatatttacagccatactagaatttttatttctacgtttttagtaaatttaatattctgggggaaaaaaggccttgattttagggttaaaaacctgacttatagaagagtttatttaatataggtcaaaattttctgtgtttcttattccttctatacctcaaatctgattctaagaatttcttactgtgataatcattggcatgccacctgaggtcaaggagtgccaaataggactttccactcatgctcaagatcaaaactttatagaacagtcaacattttagattcggtaaccttttttttcttccaattataatctctgcttctagccacttccgccagcagttggtggaagacttactaggtgcagggcactttccaagttcatcacaacaacctgcttgttttcatgagacaataatccgaaaagttcgctttgatatattcctggagggccaagcccatctatttacaaaaggtgaacagcaaaatcaagcactgctttatgggcaggaacacaagagaaagcaaactgcccaagaagtcatcatgtcagaaactcaatctcaacaaaataatttccatcagggaacttcagggtttcttgggggcttatgagtctcaccggtcaacccaggaggcctcactacaagagccttgacaaggcactgttttttgtgggactgggagttcacactgatgaagcaaacctttgaatttttgcacagctcttgtcagaaagccctgagttccccctggataaagagttaattttaatccttccctataattatacttcaaaatatttgacatctgctattatgccttctttagatctttcttctgcggtgcagacatttctagtaagtgtttgactacttgtatggcattagctttcacagaaaattgtttcacttaaaactgtggattggcctaggctaaggacaaaaataaactaagtacctgtagtgtatttatgtgatatgtgtcaagttactcaaagttattgctgttggaactgaacaataatatttcccagatagctggccttagcatgtgatcacggttgttgtatttttaatttttgtcttttacagtatgagaggtgtaggttaatttgtttatttcctataaatttgtatttatgtgtatataaaatgtacaatgaatgtaaatatgactttctggaaagtttagactacatttagaatctctattcaaaatcaaaatgctgctcaaatgaatttaaccaacatctaggtgcttaatttctcattttatcccacttatgagattgggaaaaagatcaatatgagaaataccatacagataccttaaatgtatgcatttgtgcaacaatttttgagaaggtgagtggcaatttataatttagttggcaatttataatagaacttatagcttttaaaagactttttaaagacattaaatgtaaacttaaaaatgtttagatcttgtttcaaactttacaatagcattcttcaaaatattaagttatatattttataggcatttagttgcttattaaaagcactgattttcaaactttttgatttaagaacaattatttaagatcgtctcagaagatgggatcttcgtttcaagaaaagggaatcaagtttgcctttgagataatacgttacactaagaaaaggaaaatgtggatagtaaaacccacctctctcatcctattgtactctcttctgctttttagaagcctgcacttaagcttagatttgtgaagggagagtagaaggggagaagtagaaccacagtgttttatttatttttctaaaactcttactaaatccagattttttaaactgttttaaatgtgaattcttcccagaaatttcaatgcattgcatatttagccttcggcatatttttcatgaatagatcatgaagtcataggcttccaaggcataggaagagatcttgcaggtctagtattttaataatgcactattacccagggcagatattatgagaaactgtttcttctctaagggtttatggcagactttgcttttttaacatgtgagaaatgaattttttattttgtgatttatgtgatttcttttgctgagtgaaggaaaggagaaattgttgctattgtcagcatcttaaaggtatttccagtcaaggcaaggctaagtgctttgtgatagtattaagcaagtcatgttttgaatggattacctgtagtgactcattggaatgatataattatacaagtaatgccaaaaaccaagtcaaagcctaattaaccaaagcactcatttaaaaatcatcatgtttggacctatctggacctctcagcactgtaaaatagttttggttttgtggcatatgaatagctgtttaacaaatcaaagttagctttttgcttctcagcttttttgggcaatacaagttaagttcttaatggggagacattatcatggcatgacttaagggaacattggtttgtgaaggaaaaacagattatctaaagccatctctatgtttctgttcagataaagattaatgagttctgtgtttatatcagctttgtatatttcatcttagccattctatcctagaaagattttaatgtgagcttaagatgtaaataaataattttgcaaacatgaaaaaaaaaaaaaaaaa

SEQ ID NO: 4 - protein, 2139 aa, Homo sapiens
MAEVTVPRVYVVFGIHCIMAKASSDVQVSGFHRKIQHVKNELCHMLSLEEVAPVLQQTLLQDNLLGRVHFDQFKEALILILSRTLSNEEHFQEPDCSLEAQPKYVRGGKRYGRRSLPEFQESVEEFPEVTVIEPLDEEARPSHIPAGDCSEHWKTQRSEEYEAEGQLRFWNPDDLNASQSGSSPPQDWIEEKLQEVCEDLGITRDGHLNRKKLVSICEQYGLQNVDGEMLEEVFHNLDPDGTMSVEDFFYGLFKNGKSLTPSASTPYRQLKRHLSMQSFDESGRRTTTSSAMTSTIGFRVFSCLDDGMGHASVERILDTWQEEGIENSQEILKALDFSLDGNINLTELTLALENELLVTKNSIHQAALASFKAEIRHLLERVDQVVREKEKLRSDLDKAEKLKSLMASEVDDHHAAIERRNEYNLRKLDGEYKERIAALKNELRKEREQILQQAGKQRLELEQEIEKAKTEENYIRDRLALSLKENSRLENELLENAEKLAEYENLTNKLQRNLENVLAEKFGDLDPSSAEFFLQEERLTQMRNEYERQCRVLQDQVDELQSELEEYRAQGRVLRLPLKNSPSEEVEANSGGIEPEHGLGSEECNPLNMSIEAELVIEQMKEQHHRDICCLRLELEDKVRHYEKQLDETVVSCKKAQENMKQRHENETRTLEKQISDLKNEIAELQGQAAVLKEAHHEATCRHEEEKKQLQVKLEEEKTHLQEKLRLQHEMELKARLTQAQASFEREREGLQSSAWTEEKVRGLTQELEQFHQEQLTSLVEKHTLEKEELRKELLEKHQRELQEGREKMETECNRRTSQIEAQFQSDCQKVTERCESALQSLEGRYRQELKDLQEQQREEKSQWEFEKDELTQECAEAQELLKETLKREKTTSLVLTQEREMLEKTYKEHLNSMVVERQQLLQDLEDLRNVSETQQSLLSDQILELKSSHKRELREREEVLCQAGASEQLASQRLERLEMEHDQERQEMMSKLLAMENIHKATCETADRERAEMSTEISRLQSKIKEMQQATSPLSMLQSGCQVIGEEEVEGDGALSLLQQGEQLLEENGDVLLSLQRAHEQAVKENVKMATEISRLQQRLQKLEPGLVMSSCLDEPATEFFGNTAEQTEQFLQQNRTKQVEGVTRRHVLSDLEDDEVRDLGSTGTSSVQRQEVKIEESEASVEGFSELENSEETRTESWELKNQISQLQEQLMMLCADCDRASEKKQDLLFDVSVLKKKLKMLERIPEASPKYKLLYEDVSRENDCLQEELRMMETRYDEALENNKELTAEVFRLQDELKKMEEVTETFLSLEKSYDEVKIENEGLNVLVLRLQGKIEKLQESVVQRCDCCLWEASLENLEIEPDGNILQLNQTLEECVPRVRSVHHVIEECKQENQYLEGNTQLLEKVKAHEIAWLHGTIQTHQERPRVQNQVILEENTTLLGFQDKHFQHQATIAELELEKTKLQELTRKLKERVTILVKQKDVLSHGEKEEELKAMMHDLQITCSEMQQKVELLRYESEKLQQENSILRNEITTLNEEDSISNLKLGTLNGSQEEMWQKTETVKQENAAVQKMVENLKKQISELKIKNQQLDLENTELSQKNSQNQEKLQELNQRLTEMLCQKEKEPGNSALEEREQEKFNLKEELERCKVQSSTLVSSLEAELSEVKIQTHIVQQENHLLKDELEKMKQLHRCPDLSDFQQKISSVLSYNEKLLKEKEALSEELNSCVDKLAKSSLLEHRIATMKQEQKSWEHQSASLKSQLVASQEKVQNLEDTVQNVNLQMSRMKSDLRVTQQEKEALKQEVMSLHKQLQNAGGKSWAPEIATHPSGLHNQQKRLSWDKLDHLMNEEQQLLWQENERLQTMVQNTKAELTHSREKVRQLESNLLPKHQKHLNPSGTMNPTEQEKLSLKRECDQFQKEQSPANRKVSQMNSLEQELETIHLENEGLKKKQVKLDEQLMEMQHLRSTATPSPSPHAWDLQLLQQQACPMVPREQFLQLQRQLLQAERINQHLQEELENRTSETNTPQGNQEQLVTVMEERMIEVEQKLKLVKRLLQEKVNQLKEQLCKNTKADAMVKDLYVENAQLLKALEVTEQRQKTAEKKNYLLEEKIASLSNIVRNLTPAPLTSTPPLRS

(19) United States
(12) Patent Application Publication
(10) Pub. No.: US 2003/0032592 A1
(43) Pub. Date: Feb. 13, 2003

(54) Protein-protein interactions

(76) Inventors: Daniel M. Cimbora, Salt Lake City, UT (US); Karen Heichman, Salt Lake City, UT (US); Paul L. Bartel, Salt Lake City, UT (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/023,219

(22) Filed: Dec. 20, 2001

Related U.S. Application Data
(60) Provisional application No. 60/256,983, filed on Dec. 21, 2000.

(51) Int. Cl. C07K 14/705; A61K 38/17; G01N 33/53; C07K 16/28

(52) U.S. Cl. 514/12; 530/350; 435/7.1; 530/388.1

(57) ABSTRACT

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

### CROSS REFERENCE TO RELATED APPLICATIONS

The present application is related to U.S. provisional patent application Serial No. 60/256,983, filed on Dec. 21, 2000, incorporated herein by reference, and claims priority thereto under 35 USC §119(e).

### BACKGROUND OF THE INVENTION

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

The publications and other materials used herein to illuminate the background of the invention, and in particular, cases to provide additional details respecting the practice, are incorporated herein by reference, and for convenience, are referenced by author and date in the following text and respectively grouped in the appended Bibliography.

Many processes in biology, including transcription, translation and metabolic or signal transduction pathways, are mediated by non-covalently associated protein complexes. The formation of protein-protein complexes or protein-DNA complexes produce the most efficient chemical machinery. Much of modem biological research is concerned with identifying proteins involved in cellular processes, determining their functions, and how, when and where they interact with other proteins involved in specific pathways. Further, with rapid advances in genome sequencing, there is a need to define protein linkage maps, i.e., detailed inventories of protein interactions that make up functional assemblies of proteins or protein complexes or that make up physiological pathways.

Recent advances in human genomics research has led to rapid progress in the identification of novel genes. In applications to biological and pharmaceutical research, there is a need to determine functions of gene products. A first step in defining the function of a novel gene is to determine its interactions with other gene products in appropriate context. That is, since proteins make specific interactions with other proteins or other biopolymers as part of functional assemblies or physiological pathways, an appropriate way to examine function of a gene is to determine its physical relationship with other genes. Several systems exist for identifying protein interactions and hence relationships between genes.

There continues to be a need in the art for the discovery of additional protein-protein interactions involved in mammalian physiological pathways. There continues to be a need in the art also to identify the protein-protein interactions that are involved in mammalian physiological disorders and diseases, and to thus identify drug targets.

### SUMMARY OF THE INVENTION

The present invention relates to the discovery of protein-protein interactions that are involved in mammalian physiological pathways, including physiological disorders or diseases, and to the use of this discovery. The identification of the interacting proteins described herein provide new targets for the identification of useful pharmaceuticals, new targets for diagnostic tools in the identification of individuals at risk, sequences for production of transformed cell lines, cellular models and animal models, and new bases for therapeutic intervention in such physiological pathways

Thus, one aspect of the present invention is protein complexes. The protein complexes are a complex of (a) two interacting proteins, (b) a first interacting protein and a fragment of a second interacting protein, (c) a fragment of a first interacting protein and a second interacting protein, or (d) a fragment of a first interacting protein and a fragment of a second interacting protein. The fragments of the interacting proteins include those parts of the proteins, which interact to form a complex. This aspect of the invention includes the detection of protein interactions and the production of proteins by recombinant techniques. The latter embodiment also includes cloned sequences, vectors, transfected or transformed host cells and transgenic animals.

A second aspect of the present invention is an antibody that is immunoreactive with the above complex. The antibody may be a polyclonal antibody or a monoclonal antibody. While the antibody is immunoreactive with the complex, it is not immunoreactive with the component parts of the complex. That is, the antibody is not immunoreactive with a first interactive protein, a fragment of a first interacting protein, a second interacting protein or a fragment of a second interacting protein. Such antibodies can be used to detect the presence or absence of the protein complexes.

A third aspect of the present invention is a method for diagnosing a predisposition for physiological disorders or diseases in a human or other animal. The diagnosis of such disorders includes a diagnosis of a predisposition to the disorders and a diagnosis for the existence of the disorders. In accordance with this method, the ability of a first interacting protein or fragment thereof to form a complex with a second interacting protein or a fragment thereof is assayed, or the genes encoding interacting proteins are screened for mutations in interacting portions of the protein molecules. The inability of a first interacting protein or fragment thereof to form a complex, or the presence of mutations in a gene within the interacting domain, is indicative of a predisposition to, or existence of a disorder. In accordance with one embodiment of the invention, the ability to form a complex is assayed in a two-hybrid assay. In a first aspect of this embodiment, the ability to form a complex is assayed by a yeast two-hybrid assay. In a second aspect, the ability to form a complex is assayed by a mammalian two-hybrid assay. In a second embodiment, the ability to form a complex is assayed by measuring in vitro a complex formed by combining said first protein and said second protein. In one aspect the proteins are isolated from a human or other animal. In a third embodiment, the ability to form a complex is assayed by measuring the binding of an antibody, which is specific for the complex. In a fourth embodiment, the ability to form a complex is assayed by measuring the binding of an antibody that is specific for the complex with a tissue extract from a human or other animal. In a fifth embodiment, coding sequences of the interacting proteins described herein are screened for mutations.

A fourth aspect of the present invention is a method for screening for drug candidates which are capable of modulating the interaction of a first interacting protein and a second interacting protein. In this method, the amount of the complex formed in the presence of a drug is compared with the amount of the complex formed in the absence of the drug. If the amount of complex formed in the presence of the drug is greater than or less than the amount of complex formed in the absence of the drug, the drug is a candidate for modulating the interaction of the first and second interacting proteins. The drug promotes the interaction if the complex formed in the presence of the drug is greater and inhibits (or disrupts) the interaction if the complex formed in the presence of the drug is less. The drug may affect the interaction directly, i.e., by modulating the binding of the two proteins, or indirectly, e.g., by modulating the expression of one or both of the proteins.

A fifth aspect of the present invention is a model for such physiological pathways, disorders or diseases. The model may be a cellular model or an animal model, as further described herein. In accordance with one embodiment of the invention, an animal model is prepared by creating transgenic or “knock-out” animals. The knock-out may be a total knock-out, i.e., the desired gene is deleted, or a conditional knock-out, i.e., the gene is active until it is knocked out at a determined time. In a second embodiment, a cell line is derived from such animals for use as a model. In a third embodiment, an animal model is prepared in which the biological activity of a protein complex of the present invention has been altered. In one aspect, the biological activity is altered by disrupting the formation of the protein complex, such as by the binding of an antibody or small molecule to one of the proteins which prevents the formation of the protein complex. In a second aspect, the biological activity of a protein complex is altered by disrupting the action of the complex, such as by the binding of an antibody or small molecule to the protein complex which interferes with the action of the protein complex as described herein. In a fourth embodiment, a cell model is prepared by altering the genome of the cells in a cell line. In one aspect, the genome of the cells is modified to produce at least one protein complex described herein. In a second aspect, the genome of the cells is modified to eliminate at least one protein of the protein complexes described herein.

A sixth aspect of the present invention are nucleic acids coding for novel proteins discovered in accordance with the present invention and the corresponding proteins and antibodies.

A seventh aspect of the present invention is a method of screening for drug candidates useful for treating a physiological disorder. In this embodiment, drugs are screened on the basis of the association of a protein with a particular physiological disorder. This association is established in accordance with the present invention by identifying a relationship of the protein with a particular physiological disorder. The drugs are screened by comparing the activity of the protein in the presence and absence of the drug. If a difference in activity is found, then the drug is a drug candidate for the physiological disorder. The activity of the protein can be assayed in vitro or in vivo using conventional techniques, including transgenic animals and cell lines of the present invention.

### DETAILED DESCRIPTION OF THE INVENTION

The present invention is the discovery of novel interactions between proteins described herein. The genes coding for some of these proteins may have been cloned previously, but their potential interaction in a physiological pathway or with a particular protein was unknown. Alternatively, the genes coding for some of these proteins have not been cloned previously and represent novel genes. These proteins are identified using the yeast two-hybrid method and searching a human total brain library, as more fully described below.

According to the present invention, new protein-protein interactions have been discovered. The discovery of these interactions has identified several protein complexes for each protein-protein interaction. The protein complexes for these interactions are set forth below in Tables 1-10, which also identifies the new protein-protein interactions of the present invention.

TABLE 1
Protein Complexes LXR-alpha/Utrophin Interaction

Oxysterol liver X receptor alpha (LXR-alpha) and utrophin
A fragment of LXR-alpha and utrophin
LXR-alpha and a fragment of utrophin
A fragment of LXR-alpha and a fragment of utrophin

TABLE 2
Protein Complexes LXR-alpha/zyxin Interaction

Oxysterol liver X receptor alpha (LXR-alpha) and zyxin
A fragment of LXR-alpha and zyxin
LXR-alpha and a fragment of zyxin
A fragment of LXR-alpha and a fragment of zyxin

TABLE 3
Protein Complexes LXR-alpha/LIMS1 Interaction

Oxysterol liver X receptor alpha (LXR-alpha) and LIMS1
A fragment of LXR-alpha and LIMS1
LXR-alpha and a fragment of LIMS1
A fragment of LXR-alpha and a fragment of LIMS1

TABLE 4
Protein Complexes LXR-alpha/PN7771 Interaction

Oxysterol liver X receptor alpha (LXR-alpha) and PN7771
A fragment of LXR-alpha and PN7771
LXR-alpha and a fragment of PN7771
A fragment of LXR-alpha and a fragment of PN7771

TABLE 5
Protein Complexes LXR-alpha/Homer-3 Interaction

Oxysterol liver X receptor alpha (LXR-alpha) and Homer-3
A fragment of LXR-alpha and Homer-3
LXR-alpha and a fragment of Homer-3
A fragment of LXR-alpha and a fragment of Homer-3

TABLE 6
Protein Complexes LXR-alpha/RACK1 Interaction

Oxysterol liver X receptor alpha (LXR-alpha) and RACK1
A fragment of LXR-alpha and RACK1
LXR-alpha and a fragment of RACK1
A fragment of LXR-alpha and a fragment of RACK1

TABLE 7
Protein Complexes LXR-alpha/EIF3S1 Interaction

Oxysterol liver X receptor alpha (LXR-alpha) and EIF3S1
A fragment of LXR-alpha and EIF3S1
LXR-alpha and a fragment of EIF3S1
A fragment of LXR-alpha and a fragment of EIF3S1

TABLE 8
Protein Complexes LXR-alpha/PSMD11 Interaction

Oxysterol liver X receptor alpha (LXR-alpha) and PSMD11
A fragment of LXR-alpha and PSMD11
LXR-alpha and a fragment of PSMD11
A fragment of LXR-alpha and a fragment of PSMD11

TABLE 9
Protein Complexes LXR-alpha/KIAA0610 Interaction

Oxysterol liver X receptor alpha (LXR-alpha) and KIAA0610
A fragment of LXR-alpha and KIAA0610
LXR-alpha and a fragment of KIAA0610
A fragment of LXR-alpha and a fragment of KIAA0610

TABLE 10
Protein Complexes LXR-alpha/CIR Interaction

Oxysterol liver X receptor alpha (LXR-alpha) and CIR
A fragment of LXR-alpha and CIR
LXR-alpha and a fragment of CIR
A fragment of LXR-alpha and a fragment of CIR

The involvement of above interactions in particular pathways is as follows.

Many cellular proteins exert their function by interacting with other proteins in the cell. Examples of this are found in the formation of multiprotein complexes and the association of enzymes with their substrates. It is widely believed that a great deal of information can be gained by understanding individual protein-protein interactions, and that this is useful in identifying complex networks of interacting proteins that participate in the workings of normal cellular functions. Ultimately, the knowledge gained by characterizing these networks can lead to valuable insight into the causes of human diseases and can eventually lead to the development of therapeutic strategies. The yeast two-hybrid assay is a powerful tool for determining protein-protein interactions and it has been successfully used for studying human disease pathways. In one variation of this technique, a protein of interest (or a portion of that protein) is expressed in a population of yeast cells that collectively contain all protein sequences. Yeast cells that possess protein sequences that interact with the protein of interest are then genetically selected, and the identity of those interacting proteins are determined by DNA sequencing. Thus, proteins that can be demonstrated to interact with a protein known to be involved in a human disease are therefore also implicated in that disease. Proteins identified in the first round of two-hybrid screening can be subsequently used in a second round of two-hybrid screening, allowing the identification of multiple proteins in the complex network of interactions in a disease pathway.

Nuclear hormone receptors play important roles in development, reproduction, and physiology by altering gene transcription in response to hormonal signals (Whitfield et al., 1999; Klein-Hitpass et al., 1998). Misregulation of hormone receptor signaling pathways is responsible for a variety of diseases. For example, aldosterone and its receptor (the mineralocorticoid receptor, MCR) are involved in hypertension and congestive heart failure (Duprez et al., 2000), and it has recently been shown that a missense mutation in MCR that alters its ligand specificity is responsible for pregnancy-exacerbated hypertension (Geller et al., 2000). Likewise, glucocorticoids and the glucocorticoid receptor (GR) have been implicated in chronic inflammation and arthritis (Banres, 1998), and the oxysterol liver receptor (LXR), farnesoid X receptor (FXR), and other nuclear receptors are involved in cholesterol homeostasis and atherogenesis (Schroepfer, 2000; Haynes et al., 2000; Brown and Jessup, 1999)

Collectively, the nuclear receptor superfamily is responsive to a wide variety of ligands. Nuclear hormone receptors share several important structural features, including a variable N-terminal region, a conserved central DNA-binding domain, a variable hinge region, and a conserved C-terminal ligand-binding domain (Moras and Gronemeyer, 1998; Mangelsdorf et al., 1995). Despite this conserved structural organization, interactions between ligands and receptors are remarkably specific. Hormone binding results in conformational changes in the receptor, allowing binding to specific DNA sequences (hormone response elements, HREs) in target gene promoters resulting in changes in target gene transcription. Interaction of nuclear hormone receptors with accessory proteins determines whether the receptor activates or represses transcription. Receptors can recruit coactivators that remodel chromatin and stabilize the RNA polymerase machinery, or alternatively can interact with factors that condense chromatin structure and inactivate gene expression (Wolffe et al., 1997). Furthermore, binding of a nuclear hormone receptor to other cellular proteins can alter the subcellular localization of the receptor and control its ability to bind hormone and HREs (DeFranco et al., 1998). Clearly, identification of factors with which nuclear hormone receptors interact is vital to understanding the process by which hormonal signals are transduced into transcriptional responses. In addition, identification of receptor-interacting proteins will increase the repertoire of potential targets for therapeutic intervention in the treatment of diseases due to defects involving nuclear hormone signaling.

The oxysterol liver X receptor alpha (LXRa) was used in yeast two-hybrid searches to identify novel protein-protein interactions. Here, we describe ten new interactors for LXRa. The first four interactors are involved in cell adhesion and cellular architecture. The first of these is the actin-binding protein utrophin. Utrophin is an autosomal gene that is similar to dystrophin, the gene famous for its role in Duchenne's muscular dystrophy. Unlike, dystrophin, however, utrophin appears to be expressed in a wide variety of adult tissues. Dystrophin and the dystrophin-related proteins contain spectrin repeats and likely play a role in anchoring the cytoskeleton to the plasma membrane by their actin-binding activities. The second interactor is the adhesion plaque protein zyxin, also involved in anchoring the cytoskeleton. Zyxin is a phosphoprotein that contains three LIM domains and two proline-rich regions. The interaction of LXRa with zyxin is reminiscent of the interaction we have identified between the farnesoid X-activated receptor and the LIM domain cytoskeletal protein Paxillin. Third, LXRa interacts with the novel protein PN7771, which is highly related (greater than 90% amino acid identity) to Ninein. Ninein is a centrosome-associated protein that interacts with human glycogen synthase kinase 3beta (GSK-3beta) (Hong et al., 2000), is localized to the pericentriolar matrix of the centrosome, and reacts with centrosomal autoantibody sera (Mack et al., 1998). PN7771 contains predicted calcium-binding EF hand motifs, a potential nuclear localization signal, a basic region-leucine zipper motif, a spectrin repeat, coiled-coil motifs, and Glu- and Gln-rich regions. Taken together, these interactions suggest that LXRa may be involved in cellular signaling events in response to cellular adhesion or other extracellular stimuli, and that the trans-activating ability of LXRa may be regulated by its interaction with these proteins.

Several LXRa interactors are involved in signal transduction pathways. The first is the neuronal immediate early protein homer-3. Homer proteins bind to the C-terminal tails of metabotropic glutamate receptors and play a role in their targeting and regulation; the metabotropic glutamate receptors, in turn, participate in the influx of intracellular calcium. Since LXR-alpha binds to homer-3, it is possible that LXRa may also be involved in calcium release. Alternatively, LXR-alpha could be modulated in some way by homer-3 in a manner analogous to the way in which the metabotropic glutamate receptors are regulated. The second protein, RACK1 (receptor of activated protein kinase C 1), is a WD repeat-containing protein that functions as an intracellular receptor to localize PKC to the cytoskeleton. The interaction between RACK1 and LXR-alpha suggests that LXR-alpha may be capable of localizing to the cytoskeleton via its association with RACK1. The next interactor is the LIM-domain protein LIMS 1. LIMS 1 has been implicated in integrin-linked kinase signaling, and it has been shown to interact with the SH3 and SH2 domain-containing adaptor protein NCK2 (Tu et al. 1998). Taken together, these findings suggest the involvement of LXRa in a variety of signal transduction pathways; whether LXRa activity is regulated by interaction with these proteins, or vice versa, remains to be determined.

Two LXRa interactors involved in protein metabolism were identified: the proteasome subunit PSMD11, which is involved in protein turnover, and the translation initiation factor EIF3S 1, which is involved in protein synthesis. The interaction of these proteins with LXRa suggests that nuclear hormone receptors may be involved directly with protein production and stability, in addition to transcriptional regulation.

An interaction between LXRa and the potential transmembrane protein KIAA06 10 was identified. KIAA0610 is hypothetical protein fragment 686 amino acids in length. Predicted structural motifs include four possible transmembrane domains and a coiled-coil domain. KIAA0610 displays weak homology (˜24% amino acid identity over 360-430 residues) to Drosophila and C. elegans proteins of unknown function. EST analysis suggests expression in a variety of tissues.

Finally, and interaction between LXRa and the transcription factor CIR was identified. CIR has been demonstrated to interact with the CBF1 transcription factor as well as histone deacetylase HD2 and Sin3-associated protein 30 kD (Hsieh et al., 1999). It has been proposed that CIR acts as a linker between CBF1 and the histone deacetylase complex. Similarly, the interaction of LXRa with CIR suggests CIR may link LXRa with the histone deacetylase machinery. In support of a functional role between nuclear receptors and CIR, we have also identified an interaction between CIR and the estrogen receptor ER-beta.

The proteins disclosed in the present invention were found to interact with their corresponding proteins in the yeast two-hybrid system. Because of the involvement of the corresponding proteins in the physiological pathways disclosed herein, the proteins disclosed herein also participate in the same physiological pathways. Therefore, the present invention provides a list of uses of these proteins and DNA encoding these proteins for the development of diagnostic and therapeutic tools useful in the physiological pathways. This list includes, but is not limited to, the following examples.

Two-hybrid System

The principles and methods of the yeast two-hybrid system have been described in detail elsewhere (e.g., Bartel and Fields, 1997; Bartel et al., 1993; Fields and Song, 1989; Chevray and Nathans, 1992). The following is a description of the use of this system to identify proteins that interact with a protein of interest.

The target protein is expressed in yeast as a fusion to the DNA-binding domain of the yeast Ga14p. DNA encoding the target protein or a fragment of this protein is amplified from cDNA by PCR or prepared from an available clone. The resulting DNA fragment is cloned by ligation or recombination into a DNA-binding domain vector (e.g., pGBT9, pGBT.C, pAS2-1) such that an in-frame fusion between the Ga14p and target protein sequences is created.

The target gene construct is introduced, by transformation, into a haploid yeast strain. A library of activation domain fusions (i.e., adult brain cDNA cloned into an activation domain vector) is introduced by transformation into a haploid yeast strain of the opposite mating type. The yeast strain that carries the activation domain constructs contains one or more Ga14p-responsive reporter gene(s), whose expression can be monitored. Examples of some yeast reporter strains include Y190, PJ69, and CBY14a. An aliquot of yeast carrying the target gene construct is combined with an aliquot of yeast carrying the activation domain library. The two yeast strains mate to form diploid yeast and are plated on media that selects for expression of one or more Ga14p-responsive reporter genes. Colonies that arise after incubation are selected for further characterization.

The activation domain plasmid is isolated from each colony obtained in the two-hybrid search. The sequence of the insert in this construct is obtained by the dideoxy nucleotide chain termination method. Sequence information is used to identify the gene/protein encoded by the activation domain insert via analysis of the public nucleotide and protein databases. Interaction of the activation domain fusion with the target protein is confirmed by testing for the specificity of the interaction. The activation domain construct is co-transformed into a yeast reporter strain with either the original target protein construct or a variety of other DNA-binding domain constructs. Expression of the reporter genes in the presence of the target protein but not with other test proteins indicates that the interaction is genuine.

In addition to the yeast two-hybrid system, other genetic methodologies are available for the discovery or detection of protein-protein interactions. For example, a mammalian two-hybrid system is available commercially (Clontech, Inc.) that operates on the same principle as the yeast two-hybrid system. Instead of transforming a yeast reporter strain, plasmids encoding DNA-binding and activation domain fusions are transfected along with an appropriate reporter gene (e.g., 1acZ) into a mammalian tissue culture cell line. Because transcription factors such as the Saccharomyces cerevisiae Ga14p are functional in a variety of different eukaryotic cell types, it would be expected that a two-hybrid assay could be performed in virtually any cell line of eukaryotic origin (e.g., insect cells (SF9), fungal cells, worm cells, etc.). Other genetic systems for the detection of protein-protein interactions include the so-called SOS recruitment system (Aronheim et al., 1997).

Protein-protein Interactions

Protein interactions are detected in various systems including the yeast two-hybrid system, affinity chromatography, co-immunoprecipitation, subcellular fractionation and isolation of large molecular complexes. Each of these methods is well characterized and can be readily performed by one skilled in the art. See, e.g., U.S. Pat. Nos. 5,622,852 and 5,773,218, and PCT published applications No. WO 97/27296 and WO 99/65939, each of which are incorporated herein by reference.

The protein of interest can be produced in eukaryotic or prokaryotic systems. A cDNA encoding the desired protein is introduced in an appropriate expression vector and transfected in a host cell (which could be bacteria, yeast cells, insect cells, or mammalian cells). Purification of the expressed protein is achieved by conventional biochemical and immunochemical methods well known to those skilled in the art. The purified protein is then used for affinity chromatography studies: it is immobilized on a matrix and loaded on a column. Extracts from cultured cells or homogenized tissue samples are then loaded on the column in appropriate buffer, and non-binding proteins are eluted. After extensive washing, binding proteins or protein complexes are eluted using various methods such as a gradient of pH or a gradient of salt concentration. Eluted proteins can then be separated by two-dimensional gel electrophoresis, eluted from the gel, and identified by micro-sequencing. The purified proteins can also be used for affinity chromatography to purify interacting proteins disclosed herein. All of these methods are well known to those skilled in the art.

Similarly, both proteins of the complex of interest (or interacting domains thereof) can be produced in eukaryotic or prokaryotic systems. The proteins (or interacting domains) can be under control of separate promoters or can be produced as a fusion protein. The fusion protein may include a peptide linker between the proteins (or interacting domains) which, in one embodiment, serves to promote the interaction of the proteins (or interacting domains). All of these methods are also well known to those skilled in the art.

Purified proteins of interest, individually or a complex, can also be used to generate antibodies in rabbit, mouse, rat, chicken, goat, sheep, pig, guinea pig, bovine, and horse. The methods used for antibody generation and characterization are well known to those skilled in the art. Monoclonal antibodies are also generated by conventional techniques. Single chain antibodies are further produced by conventional techniques.

DNA molecules encoding proteins of interest can be inserted in the appropriate expression vector and used for transfection of eukaryotic cells such as bacteria, yeast, insect cells, or mammalian cells, following methods well known to those skilled in the art. Transfected cells expressing both proteins of interest are then lysed in appropriate conditions, one of the two proteins is immunoprecipitated using a specific antibody, and analyzed by polyacrylamide gel electrophoresis. The presence of the binding protein (co-immunoprecipitated) is detected by immunoblotting using an antibody directed against the other protein. Co-immunoprecipitation is a method well known to those skilled in the art.

Transfected eukaryotic cells or biological tissue samples can be homogenized and fractionated in appropriate conditions that will separate the different cellular components. Typically, cell lysates are run on sucrose gradients, or other materials that will separate cellular components based on size and density. Subcellular fractions are analyzed for the presence of proteins of interest with appropriate antibodies, using immunoblotting or immunoprecipitation methods. These methods are all well known to those skilled in the art.

Disruption of Protein-protein Interactions

It is conceivable that agents that disrupt protein-protein interactions can be beneficial in many physiological disorders, including, but not-limited to NIDDM, AD and others disclosed herein. Each of the methods described above for the detection of a positive protein-protein interaction can also be used to identify drugs that will disrupt said interaction. As an example, cells transfected with DNAs coding for proteins of interest can be treated with various drugs, and co-immunoprecipitations can be performed. Alternatively, a derivative of the yeast two-hybrid system, called the reverse yeast two-hybrid system (Leanna and Hannink, 1996), can be used, provided that the two proteins interact in the straight yeast two-hybrid system.

Modulation of Protein-protein Interactions

Since the interactions described herein are involved in a physiological pathway, the identification of agents which are capable of modulating the interactions will provide agents which can be used to track physiological disorder or to use lead compounds for development of therapeutic agents. An agent may modulate expression of the genes of interacting proteins, thus affecting interaction of the proteins. Alternatively, the agent may modulate the interaction of the proteins. The agent may modulate the interaction of wild-type with wild-type proteins, wild-type with mutant proteins, or mutant with mutant proteins. Agents which may be used to modulate the protein interaction include a peptide, an antibody, a nucleic acid, an antisense compound or a ribozyme. The nucleic acid may encode the antibody or the antisense compound. The peptide may be at least 4 amino acids of the sequence of either of the interacting proteins. Alternatively, the peptide may be from 4 to 30 amino acids (or from 8 to 20 amino acids) that is at least 75% identical to a contiguous span of amino acids of either of the interacting proteins. The peptide may be covalently linked to a transporter capable of increasing cellular uptake of the peptide. Examples of a suitable transporter include penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histine oligomers, D-histine oligomers, L-ornithine oligomers, D-ornithine oligomers, short peptide sequences derived from fibroblast growth factor, Galparan, and HSV-1 structural protein VP22, and peptoid analogs thereof. Agents can be tested using transfected host cells, cell lines, cell models or animals, such as described herein, by techniques well known to those of ordinary skill in the art, such as disclosed in U.S. Pat. Nos. 5,622,852 and 5,773,218, and PCT published application Nos. WO 97/27296 and WO 99/65939, each of which are incorporated herein by reference. The modulating effect of the agent can be tested in vivo or in vitro. Agents can be provided for testing in a phage display library or a combinatorial library. Exemplary of a method to screen agents is to measure the effect that the agent has on the formation of the protein complex.

Mutation Screening

The proteins disclosed in the present invention interact with one or more proteins known to be involved in a physiological pathway, such as in NIDDM, AD or pathways described herein. Mutations in interacting proteins could also be involved in the development of the physiological disorder, such as NIDDM, AD or disorders described herein, for example, through a modification of protein-protein interaction, or a modification of enzymatic activity, modification of receptor activity, or through an unknown mechanism. Therefore, mutations can be found by sequencing the genes for the proteins of interest in patients having the physiological disorder, such as insulin, and non-affected controls. A mutation in these genes, especially in that portion of the gene involved in protein interactions in the physiological pathway, can be used as a diagnostic tool and the mechanistic understanding the mutation provides can help develop a therapeutic tool.

Screening for At-risk Individuals

Individuals can be screened to identify those at risk by screening for mutations in the protein disclosed herein and identified as described above. Alternatively, individuals can be screened by analyzing the ability of the proteins of said individual disclosed herein to form natural complexes. Further, individuals can be screened by analyzing the levels of the complexes or individual proteins of the complexes or the MRNA encoding the protein members of the complexes. Techniques to detect the formation of complexes, including those described above, are known to those skilled in the art. Techniques and methods to detect mutations are well known to those skilled in the art. Techniques to detect the level of the complexes, proteins or mRNA are well known to those skilled in the art.

Cellular Models of Physiological Disorders

A number of cellular models of many physiological disorders or diseases have been generated. The presence and the use of these models are familiar to those skilled in the art. As an example, primary cell cultures or established cell lines can be transfected with expression vectors encoding the proteins of interest, either wild-type proteins or mutant proteins. The effect of the proteins disclosed herein on parameters relevant to their particular physiological disorder or disease can be readily measured. Furthermore, these cellular systems can be used to screen drugs that will influence those parameters, and thus be potential therapeutic tools for the particular physiological disorder or disease. Alternatively, instead of transfecting the DNA encoding the protein of interest, the purified protein of interest can be added to the culture medium of the cells under examination, and the relevant parameters measured.

Animal Models

The DNA encoding the protein of interest can be used to create animals that overexpress said protein, with wild-type or mutant sequences (such animals are referred to as “transgenic”), or animals which do not express the native gene but express the gene of a second animal (referred to as “transplacement”), or animals that do not express said protein (referred to as “knock-out”). The knock-out animal may be an animal in which the gene is knocked out at a determined time. The generation of transgenic, transplacement and knock-out animals (normal and conditioned) uses methods well known to those skilled in the art.

In these animals, parameters relevant to the particular physiological disorder can be measured. These parametes may include receptor function, protein secretion in vivo or in vitro, survival rate of cultured cells, concentration of particular protein in tissue homogenates, signal transduction, behavioral analysis, protein synthesis, cell cycle regulation, transport of compounds across cell or nuclear membranes, enzyme activity, oxidative stress, production of pathological products, and the like. The measurements of biochemical and pathological parameters, and of behavioral parameters, where appropriate, are performed using methods well known to those skilled in the art. These transgenic, transplacement and knock-out animals can also be used to screen drugs that may influence the biochemical, pathological, and behavioral parameters relevant to the particular physiological disorder being studied. Cell lines can also be derived from these animals for use as cellular models of the physiological disorder, or in drug screening.

Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art. Such techniques may include providing atomic coordinates defining a three-dimensional structure of a protein complex formed by said first polypeptide and said second polypeptide, and designing or selecting compounds capable of interfering with the interaction between a first polypeptide and a second polypeptide based on said atomic coordinates.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Diagnostic Assays

The identification of the interactions disclosed herein enables the development of diagnostic assays and kits, which can be used to determine a predisposition to or the existence of a physiological disorder. In one aspect, one of the proteins of the interaction is used to detect the presence of a “normal” second protein (i.e., normal with respect to its ability to interact with the first protein) in a cell extract or a biological fluid, and further, if desired, to detect the quantitative level of the second protein in the extract or biological fluid. The absence of the “normal” second protein would be indicative of a predisposition or existence of the physiological disorder. In a second aspect, an antibody against the protein complex is used to detect the presence and/or quantitative level of the protein complex. The absence of the protein complex would be indicative of a predisposition or existence of the physiological disorder.

Nucleic Acids and Proteins

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, more preferably at least about 95% of the nucleotide bases, and more preferably at least about 98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more usually at least about 80% identity, preferably at least about 90% identity, more preferably at least about 95% identity, and most preferably at least about 98% identity.

Identity means the degree of sequence relatedness between two polypeptide or two polynucleotides sequences as determined by the identity of the match between two strings of such sequences. Identity can be readily calculated. While there exist a number of methods to measure identity between two polynucleotide or polypeptide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48:1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1).387 (1984)), BLASTP, BLASTN, FASTA (Altschul et al. (1990); Altschul et al. (1997)). The well-known Smith Waterman algorithm may also be used to determine identity.

Alternatively, substantial homology or similarity exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. See, e.g., Asubel, 1992; Wetmur and Davidson, 1968.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animals or (b) chemical synthesis using techniques well known in the art. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the art.

The nucleic acid or protein may also be incorporated on a microarray. The preparation and use of microarrays are well known in the art. Generally, the microarray may contain the entire nucleic acid or protein, or it may contain one or more fragments of the nucleic acid or protein. Suitable nucleic acid fragments may include at least 17 nucleotides, at least 21 nucleotides, at least 30 nucleotides or at least 50 nucleotides of the nucleic acid sequence, particularly the coding sequence. Suitable protein fragments may include at least 4 amino acids, at least 8 amino acids, at least 12 amino acids, at least 15 amino acids, at least 17 amino acids or at least 20 amino acids. Thus, the present invention is also directed to such nucleic acid and protein fragments.

### EXAMPLES

The present invention is further detailed in the following Examples, which are offered by way of illustration and are not intended to limit the invention in any manner. Standard techniques well known in the art or the techniques specifically described below are utilized.

### Example 1

### Yeast Two-hybrid System

The principles and methods of the yeast two-hybrid systems have been described in detail (Bartel and Fields, 1997). The following is thus a description of the particular procedure that we used, which was applied to all proteins.

The cDNA encoding the bait protein was generated by PCR from brain cDNA. Gene-specific primers were synthesized with appropriate tails added at their 5′ ends to allow recombination into the vector pGBTQ. The tail for the forward primer was 5′-GCAGGAAACAGCTATGACCATACAGTCAGCGGCCGCCACC-3′ (SEQ ID NO: 1) and the tail for the reverse primer was 5′-ACGGCCAGTCGCGTGGAGTGTTATGTCATGCGGCCGCTA-3′ (SEQ ID NO: 2). The tailed PCR product was then introduced by recombination into the yeast expression vector PGBTQ, which is a close derivative of pGBTC (Bartel et al., 1996) in which the polylinker site has been modified to include M13 sequencing sites. The new construct was selected directly in the yeast J693 for its ability to drive tryptophane synthesis (genotype of this strain: Mat α, ade2, his3, leu2, trp1, URA3::GAL1-1acZ LYS2::GAL1-HIS3 ga14del ga180del cyhR2). In these yeast cells, the bait is produced as a C-terminal fusion protein with the DNA binding domain of the transcription factor Ga14 (amino acids 1 to 147). A total human brain (37 year-old male Caucasian) cDNA library cloned into the yeast expression vector pACT2 was purchased from Clontech (human brain MATCHMAKER cDNA, cat. # HL4004AH), transformed into the yeast strain J692 (genotype of this strain: Mat a, ade2, his3, leu2, trp1, URA3::GAL1-1acZ LYS2::GAL1-HIS3 ga14del ga180del cyhR2), and selected for the ability to drive leucine synthesis. In these yeast cells, each cDNA is expressed as a fusion protein with the transcription activation domain of the transcription factor Ga14 (amino acids 768 to 881) and a 9 amino acid hemagglutinin epitope tag. J693 cells (Mat α type) expressing the bait were then mated with J692 cells (Mat α type) expressing proteins from the brain library. The resulting diploid yeast cells expressing proteins interacting with the bait protein were selected for the ability to synthesize tryptophan, leucine, histidine, and β-galactosidase. DNA was prepared from each clone, transformed by electroporation into E. coli strain KC8 (Clontech KC8 electrocompetent cells, cat. # C2023-1), and the cells were selected on ampicillin-containing plates in the absence of either tryptophane (selection for the bait plasmid) or leucine (selection for the brain library plasmid). DNA for both plasmids was prepared and sequenced by di-deoxynucleotide chain termination method. The identity of the bait cDNA insert was confirmed and the cDNA insert from the brain library plasmid was identified using BLAST program against public nucleotides and protein databases. Plasmids from the brain library (preys) were then individually transformed into yeast cells together with a plasmid driving the synthesis of lamin fused to the Ga14 DNA binding domain. Clones that gave a positive signal after β-galactosidase assay were considered false-positives and discarded. Plasmids for the remaining clones were transformed into yeast cells together with plasmid for the original bait. Clones that gave a positive signal after β-galactosidase assay were considered true positives.

### Example 2

Identification of LXR-alpha/Utrophin Interaction

A yeast two-hybrid system as described in Example 1 using amino acids 95-277 of LXR-alpha (GenBank (GB) accession no. U22662) as bait was performed. One clone that was identified by this procedure included amino acids 2443-2650 of utophin (GB accession no. X15488).

### Example 3

### Identification of LXR-alpha/PN7771 Interaction

A yeast two-hybrid system as described in Example 1 using amino acids 95-277 of LXR-alpha (GB accession no. U22662) as bait was performed. One clone that was identified by this procedure included amino acids 1747-2047 of PN7771. The DNA sequence and the predicted protein sequence for PN7771 are set forth in Tables 11 and 12, respectively.

TABLE 11
Nucleotide Sequence of PN7771

| | |
|---|---|
| cttattttgaaaacatttacatagtgattagttaacccaacagaccaatcctgggaagacagccagagcctgcagcaccttagtaacaga | (SEQ ID NO:3) |
| aaaactgataattaggagaagagacctgtccaagaccaggaacctggaccaaaattgtgccatgttgctttactttaatgagtggcccca | |
| gtaaaaactgagctgtatggcagagctgttcacatttatcttctgtgtccacccagttctgctgaaacccctggcaagatcgtggccctg | |
| ttgtagcttgtcatgttttgaacagctgtctatggaaagaaagcaaacacaacctagagcaacattgatttgttttagaaagctctttta | |
| ttttcagttctggctgtgttcaacatcttagcttacgtttttcatgttgtaatgatctgccgtatggacgatcacctctaagttagagag | |
| ttctgtaatttggcttggattaaagatgcttggttagtgaaagctgctgctttttttatagtcaaaggactggttctgagagccttgttg | |
| cagatggctgaggtcaccgtcccaagggtgtatgtcgtgtttggcatccattgcatcatggcgaaggcatcttcagatgtgcaggtttca | |
| ggctttcatcggaaaatccagcacgttaaaaatgaactttgccacatgttgagcttggaggaggtggccccagtgctgcagcagacatta | |
| cttcaggacaacctcttgggcagggtacattttgaccaatttaaagaagcattaatactcatcttgtccagaactctgtcaaatgaagaa | |
| cactttcaagaaccagactgctcactagaagctcagcccaaatatgttagaggtgggaagcgttacggacgaaggtccttgcccgagttc | |
| caagagtccgtggaggagtttcctgaagtgacggtgattgagccactggatgaagaagcgcggccttcacacatcccagccggtgactgc | |
| agtgagcactggaagacgcaacgcagtgaggagtatgaagcggaaggccagttaaggttttggaacccagatgacttgaatgcttcacag | |
| agtggatcttcccctccccaagactggatagaagagaaactgcaagaagtttgtgaagatttggggatcacccgtgatggtcacctgaac | |
| cggaagaagctggtctccatctgtgagcagtatggtttacagaatgtggatggagagatgctcgaggaagtattccataatcttgatcct | |
| gacggtacaatgagtgtagaagattttttctatggtttgtttaaaaatggaaaatctcttacaccatcagcatctactccatatagacaa | |
| ctaaaaaggcacctttccatgcagtctttcgatgagagtggacgacgtaccacaacctcatcagcaatgacaagtaccattggctttcgg | |
| gtcttctcctgcctggatgatgggatgggccatgcatctgtggagagaatactggacacctggcaggaagagggcattgagaacagccag | |
| gagatcctgaaggccttggatttcagcctcgatggaaacatcaatttgacagaattaacactggcccttgaaaatgaacttttggttacc | |
| aagaacagcattcaccaggcggctctggccagctttaaggctgaaatccggcatttgttggaacgagttgatcaggtggtcagagaaaaa | |
| gagaagctacggtcagatctggacaaggccgagaagctcaagtctttaatggcctcggaggtggatgatcaccatgcggccatagagcgg | |
| cggaatgagtacaacctcaggaaactggatggagagtacaaggagcgaatagcagccttaaaaaatgaactccgaaaagagagagagcag | |
| atcctgcagcaggcaggcaagcagcgtttagaacttgaacaggaaattgaaaaggcaaaaacagaagagaactatatccgggaccgcctt | |
| gccctctctttaaaggaaaacagtcgtctggaaaatgagcttctagaaaatgcagagaagttggcagaatatgagaatctgacaaacaaa | |
| cttcagagaaatttggaaaatgtgttagcagaaaagtttggtgacctcgatcctagcagtgctgagttcttcctgcaagaagagagactg | |
| acacagatgagaaatgaatatgagcggcagtgcagggtactacaagaccaagtagatgaactccagtctgagctggaagaatatcgtgca | |
| caaggcagagtgctcaggcttccgttgaagaactcaccgtcagaagaagttgaggctaacagcggtggcattgagcccgaacacgggctc | |
| ggttctgaagaatgcaatccattgaatatgagcattgaggcagagctggtcattgaacagatgaaagaacaacatcacagggacatatgt | |
| tgcctcagactggagctcgaagataaagtgcgccattatgaaaagcagctggacgaaaccgtggtcagctgcaagaaggcacaggagaac | |
| atgaagcaaaggcatgagaacgaaacgcgcaccttagaaaaacaaataagtgaccttaaaaatgaaattgctgaacttcaggggcaagca | |
| gcagtgctcaaggaggcacatcatgaggccacttgcaggcatgaggaggagaaaaaacaactgcaagtgaagcttgaggaggaaaagact | |
| cacctgcaggagaagctgaggctgcaacatgagatggagctcaaggctagactgacacaggctcaagcaagctttgagcgggagagggaa | |
| ggccttcagagtagcgcctggacagaagagaaggtgagaggcttgactcaggaactagagcagtttcaccaggagcagctgacaagcctg | |
| gtggagaaacacactcttgagaaagaggagttaagaaaagagctcttggaaaagcaccaaagggagcttcaggagggaagggaaaaaatg | |
| gaaacagagtgtaatagaagaacctctcaaatagaagcccagtttcagtctgattgtcagaaagtcactgagaggtgtgaaagcgctctg | |
| caaagcctggaggggcgctaccgccaagagctgaaggacctccaggaacagcagcgtgaggagaaatcccagtgggaatttgagaaggac | |
| gagctcacccaggagtgtgcggaagcccaggagctgctgaaagagactcttaagagagagaaaacaacttctctggtcctgacccaggag | |
| agagagatgctggagaaaacatacaaagaacatttgaacagcatggtcgtcgagagacagcagctactccaagacctggaagacctaaga | |
| aatgtatctgaaacccagcaaagcctgctgtctgaccagatacttgagctgaagagcagtcacaaaagggaactgagggagcgtgaggag | |
| gtcctgtgccaggcaggggcttcggagcagctggccagccagcggctggaaagactagaaatggaacatgaccaggaaaggcaggaaatg | |
| atgtccaagcttctagccatggagaacattcacaaagcgacctgtgagacagcagatcgagaaagagccgagatgagcacagaaatctcc | |
| agacttcagagtaaaataaaggaaatgcagcaggcaacatctcctctctcaatgcttcagagtggttgccaggtgataggagaggaggag | |
| gtggaaggagatggagccctgtccctgcttcagcaaggggagcagctgttggaagaaaatggggacgtcctcttaagcctgcagagagct | |
| catgaacaggcagtgaaggaaaatgtgaaaatggctactgaaatttctagattgcaacagaggctacaaaagttagagccagggttagta | |
| atgtcttcttgtttggatgagccagctactgagttttttggaaatactgcggaacaaacagagcagtttttacagcaaaaccgaacgaag | |
| caagtagaaggtgtgaccaggcggcatgtcctaagtgacctggaagatgatgaggtccgggacctgggaagtacagggacgagctctgtt | |
| cagagacaggaagtcaaaatagaggagtctgaagcttcagtagagggtttttctgagcttgaaaacagtgaagagaccaggactgaatcc | |
| tgggagctgaagaatcagattagtcagcttcaggaacagctaatgatgttatgtgcggactgtgatcgagcttctgaaaagaaacaggac | |
| ctactttttgatgtttctgtgctaaaaaagaaactgaagatgcttgagagaatccctgaggcttctcccaaatataagctgttgtatgaa | |
| gatgtgagccgagaaaatgactgccttcaggaagagctgagaatgatggagacacgctacgatgaggcactagaaaataacaaagaactc | |
| actgcagaggttttcaggttgcaggatgagctgaagaaaatggaggaagtcactgaaacattcctcagcctggaaaagagttacgatgag | |
| gtcaaaatagaaaatgaggggctgaatgttctggttttgagacttcaaggcaagattgagaagcttcaggaaagcgtggtccagcggtgt | |
| gactgctgcttatgggaagccagtttagagaacctggaaatcgaacctgatggaaatatactccagctcaatcagacactggaagagtgt | |
| gtgcccagggttaggagtgtacatcatgtcatagaggaatgtaagcaagaaaaccagtaccttgaggggaacacacagctcttggaaaaa | |
| gtaaaagcacatgaaattgcctggttacatggaacaattcagacacatcaagaaaggccaagagtacagaatcaagttatactggaggaa | |
| aacactactctcctaggctttcaagacaaacattttcagcatcaggccaccatagcagagttagaactggagaaaacaaagttacaggag | |
| ctgactaggaagttgaaggagagagtcactattttagttaagcaaaaagatgtactttctcacggagaaaaggaggaagagctgaaggca | |
| atgatgcatgacttgcagatcacgtgcagtgagatgcagcaaaaagttgaacttctgagatatgaatctgaaaagcttcaacaggaaaat | |
| tctattttgagaaatgaaattactactttaaatgaagaagatagcatttctaacctgaaattagggacattaaatggatctcaggaagaa | |
| atgtggcaaaaaacggaaactgtaaaacaagaaaatgctgcagttcagaagatggttgaaaatttaaagaaacagatttcagaattaaaa | |
| atcaaaaaccaacaattggatttggaaaatacagaacttagccaaaagaactctcaaaaccaggaaaaactgcaagaacttaatcaacgt | |
| ctaacagaaatgctatgccagaaggaaaaagagccaggaaacagtgcattggaggaacgggaacaagagaagtttaatctgaaagaagaa | |
| ctggaacgttgtaaagtgcagtcctccactttagtgtcttctctggaggcggagctctctgaagttaaaatacagacccatattgtgcaa | |
| caggaaaaccaccttctcaaagatgaactggagaaaatgaaacagctgcacagatgtcccgatctctctgacttccagcaaaaaatctct | |
| agtgttctaagctacaacgaaaaactgctgaaagaaaaggaagctctgagtgaggaattaaatagctgtgtcgataagttggcaaaatca | |
| agtcttttagagcatagaattgcgacgatgaagcaggaacagaaatcctgggaacatcagagtgcgagcttaaagtcacagctggtggct | |
| tctcaggaaaaggttcagaatttagaagacaccgtgcagaatgtaaacctgcaaatgtcccggatgaaatctgacctacgagtgactcag | |
| caggaaaaggaggctttaaaacaagaagtgatgtctttacataagcaacttcagaatgctggtggcaagagctgggccccagagatagct | |
| actcatccatcagggctccataaccagcagaaaaggctgtcctgggacaagttggatcatctgatgaatgaggaacagcagctgctttgg | |
| caagagaatgagaggctccagaccatggtacagaacaccaaagccgaactcacgcactcccgggagaaggtccgtcaattggaatccaat | |
| cttcttcccaagcaccaaaacatctaaacccatcaggtaccatgaatcccacagagcaagaaaaattgagcttaaagagagagtgtgatc | |
| agtttcagaaagaacaatctcctgctaacaggaaggtcagtcagatgaattcccttgaacaagaattagaaacaattcatttggaaaatg | |
| aaggcctgaaaaagaaacaagtaaaactggatgagcagctcatggagatgcagcacctgaggtccactgcgacgcctagcccgtcccctc | |
| atgcttgggatttgcagctgctccagcagcaagcctgtccgatggtgcccagggagcagtttctgcagcttcaacgccagctgctgcagg | |
| cagaaaggataaaccagcacctgcaggaggaacttgaaaacaggacctccgaaaccaacacaccacagggaaaccaggaacaactggtaa | |
| ctgtcatggaggaacgaatgatagaagttgaacagaaactgaaactagtgaaaaggcttcttcaagagaaagtgaatcagctcaaagaac | |
| aactctgcaagaacactaaggcagacgcaatggtgaaggacttgtatgttgaaaatgcccagttgttgaaagctctggaagtgactgaac | |
| agcgacagaaaacagcagagaagaaaaattacctcctggaggagaagattgccagcctcagtaatatagttaggaatctgacaccagcgc | |
| cattgacttctacacctcctttgaggtcatagccaaaccaaagggtacactcatatttgtgcactttactgaaatagatgaacatttcag | |
| taggttctcaacttaaaattaagcctaacctaaaactgccagcaacacaactggagtttccatttatcataattagtttttctaaataga | |
| cccttatgggagtttgaaaataaatactcacatatttcactacttaaattattcccaagatttgaatttattttaaaattttaatagcca | |
| ccaagaatgtggacatatgaaaattcaagaacctaaaaaataccagttttgaatgagtttttgtggttttggttttttaattattacaaa | |
| tctatgtgtaaaatctagatatttgaagtttgagatctgatgagaatggttgttataaactttattttaaaaccaaatttaggtgttctt | |
| acatatttaaatactggaaagtcattataatagttttggttctttgaattggtagacaattagtagagtataattggttaggaggcaggg | |
| cttattaagtggttattaaccgctgacatcagacaaacccaaatctgtagaattctaacctcctaacacctgtgacagtattaccactct | |
| tcttgtattatagatttagaactgatttactcaattgcactcttaactaatgttaaaagcttacttgctttaaacagccttttcttcttt | |
| ctcttaaaagtttcatttggggagctggtcttctaagaaacggataaagccacataattaaagcagttgaactagagggaaagcactgaa | |
| caaaccactttggagtaaatagctactcttagaaaagagggataagcagaccatgtaggttttctgtctctcaaatcttagagttcataa | |
| atttacttgaggttgcctcaagaactcagggaacaatactgtaaactgtcttcctgaactactgtagggcctctctaagaatttgaaatg | |
| tataaaccatgtgacctcatttatttgtcttatatatttacagccatactagaatttttatttctacgtttttagtaaatttaatattct | |
| gggggaaaaaaggccttgattttagggttaaaaacctgacttatagaagagtttatttaatataggtcaaaattttctgtgtttcttatt | |
| ccttctatacctcaaatctgattctaagaatttcttactgtgataatcattggcatgccacctgaggtcaaggagtgccaaataggactt | |
| tccactcatgctcaagatcaaaactttatagaacagtcaacattttagattcggtaaccttttttttcttccaattataatctctgcttc | |
| tagccacttccgccagcagttggtggaagacttactaggtgcagggcactttccaagttcatcacaacaacctgcttgttttcatgagac | |
| aataatccgaaaagttcgctttgatatattcctggagggccaagcccatctatttacaaaaggtgaacagcaaaatcaagcactgcttta | |
| tgggcaggaacacaagagaaagcaaactgcccaagaagtcatcatgtcagaaactcaatctcaacaaaataatttccatcagggaacttc | |
| agggtttcttgggggcttatgagtctcaccggtcaacccaggaggcctcactacaagagccttgacaaggcactgttttttgtgggactg | |
| ggagttcacactgatgaagcaaacctttgaatttttgcacagctcttgtcagaaagccctgagttccccctggataaagagttaatttta | |
| atccttccctataattatacttcaaaatatttgacatctgctattatgccttctttagatctttcttctgcggtgcagacatttctagta | |
| agtgtttgactacttgtatggcattagctttcacagaaaattgtttcacttaaaactgtggattggcctaggctaaggacaaaaataaac | |
| taagtacctgtagtgtatttatgtgatatgtgtcaagttactcaaagttattgctgttggaactgaacaataatatttcccagatagctg | |
| gccttagcatgtgatcacggttgttgtatttttaatttttgtcttttacagtatgagaggtgtaggttaatttgtttatttcctataaat | |
| ttgtatttatgtgtatataaaatgtacaatgaatgtaaatatgactttctggaaagtttagactacatttagaatctctattcaaaatca | |
| aaatgctgctcaaatgaatttaaccaacatctaggtgcttaatttctcattttatcccacttatgagattgggaaaaagatcaatatgag | |
| aaataccatacagataccttaaatgtatgcatttgtgcaacaatttttgagaaggtgagtggcaatttataatttagttggcaatttata | |
| atagaacttatagcttttaaaagactttttaaagacattaaatgtaaacttaaaaatgtttagatcttgtttcaaactttacaatagcat | |
| tcttcaaaatattaagttatatattttataggcatttagttgcttattaaaagcactgattttcaaactttttgatttaagaacaattat | |
| ttaagatcgtctcagaagatgggatcttcgtttcaagaaaagggaatcaagtttgcctttgagataatacgttacactaagaaaaggaaa | |
| atgtggatagtaaaacccacctctctcatcctattgtactctcttctgctttttagaagcctgcacttaagcttagatttgtgaagggag | |
| agtagaaggggagaagtagaaccacagtgttttatttatttttctaaaactcttactaaatccagattttttaaactgttttaaatgtga | |
| attcttcccagaaatttcaatgcattgcatatttagccttcggcatatttttcatgaatagatcatgaagtcataggcttccaaggcata | |
| ggaagagatcttgcaggtctagtattttaataatgcactattacccagggcagatattatgagaaactgtttcttctctaagggtttatg | |
| gcagactttgcttttttaacatgtgagaaatgaattttttattttgtgatttatgtgatttcttttgctgagtgaaggaaaggagaaatt | |
| gttgctattgtcagcatcttaaaggtatttccagtcaaggcaaggctaagtgctttgtgatagtattaagcaagtcatgttttgaatgga | |
| ttacctgtagtgactcattggaatgatataattatacaagtaatgccaaaaaccaagtcaaagcctaattaaccaaagcactcatttaaa | |
| aatcatcatgtttggacctatctggacctctcagcactgtaaaatagttttggttttgtggcatatgaatagctgtttaacaaatcaaag | |
| ttagctttttgcttctcagcttttttgggcaatacaagttaagttcttaatggggagacattatcatggcatgacttaagggaacattgg | |
| tttgtgaaggaaaaacagattatctaaagccatctctatgtttctgttcagataaagattaatgagttctgtgtttatatcagctttgta | |
| tatttcatcttagccattctatcctagaaagattttaatgtgagcttaagatgtaaataaataattttgcaaacatgaaaaaaaaaaaaa | |
| aaaa | |

TABLE 12
Predicted Amino Acid Sequence of PN7771

| | |
|---|---|
| MAEVTVPRVYVVFGIHCIMAKASSDVQVSGFHRKIQHVKNELCHMLSLEEVAPVLQQTLL | (SEQ ID NO:4) |
| QDNLLGRVHFDQFKEALILILSRTLSNEEHFQEPDCSLEAQPKYVRGGKRYGRRSLPEFQ | |
| ESVEEFPEVTVIEPLDEEARPSHIPAGDCSEHWKTQRSEEYEAEGQLRFWNPDDLNASQS | |
| GSSPPQDWIEEKLQEVCEDLGITRDGHLNRKKLVSICEQYGLQNVDGEMLEEVFHNLDPD | |
| GTMSVEDFFYGLFKNGKSLTPSASTPYRQLKRHLSMQSFDESGRRTTTSSAMTSTIGFRV | |
| FSCLDDGMGHASVERILDTWQEEGIENSQEILKALDFSLDGNINLTELTLALENELLVTK | |
| NSIHQAALASFKAEIRHLLERVDQVVREKEKLRSDLDKAEKLKSLMASEVDDHHAAIERR | |
| NEYNLRKLDGEYRERIAALKNELRKEREQILQQAGKQRLELEQEIEKAKTEENYIRDRLA | |
| LSLKENSRLENELLENAEKLAEYENLTNKLQRNLENVLAEKFGDLDPSSAEFFLQEERLT | |
| QMRNEYERQCRVLQDQVDELQSELEEYRAQGRVLRLPLKNSPSEEVEANSGGIEPEHGLG | |
| SEECNPLNMSIEAELVIEQMKEQHHRDICCLRLELEDKVRHYEKQLDETVVSCKKAQENM | |
| KQRHENETRTLEKQISDLKNEIAELQGQAAVLKEAHHEATCRHEEEKKQLQVKLEEEKTH | |
| LQEKLRLQHEMELKARLTQAQASFEREREGLQSSAWTEEKVRGLTQELEQFHQEQLTSLV | |
| EKHTLEKEELRKELLEKHQRELQEGREKMETECNRRTSQIEAQFQSDCQKVTERCESALQ | |
| SLEGRYRQELKDLQEQQREEKSQWEFEKDELTQECAEAQELLKETLKREKTTSLVLTQER | |
| EMLEKTYKEHLNSMVVERQQLLQDLEDLRNVSETQQSLLSDQILELKSSHKRELREREEV | |
| LCQAGASEQLASQRLERLEMEHDQERQEMMSKLLAMENIHIKATCETADRERAEMSTEIS | |
| RLQSKIKEMQQATSPLSMLQSGCQVIGEEEVEGDGALSLLQQGEQLLEENGDVLLSLQRA | |
| HEQAVKENVKMATEISRLQQRLQKLEPGLVMSSCLDEPATEFFGNTAEQTEQFLQQNRTK | |
| QVEGVTRRHVLSDLEDDEVRDLGSTGTSSVQRQEVKIEESEASVEGFSELENSEETRTES | |
| WELKNQISQLQEQLMMLCADCDRASEKKQDLLFDVSVLKKKLKMLERIPEASPKYKLLYE | |
| DVSRENDCLQEELRMMETYRYDEALENNKELTAEVFRLQDELKKMEEVTETFLSLEKSYD | |
| EVKIENEGLNVLVLRLQGKIEKLQESVVQRCDCCLWEASLENLEIEPDGNILQLNQTLEE | |
| CVPRVRSVHHVIEECKQENQYLEGNTQLLEKVKAHEIAWLHGTIQTHQERPRVQNQVILE | |
| ENTTLLGFQDKHFQHQATIAELELEKTKLQELTRKLKERVTILVKQKDVLSHGEKEEELK | |
| AMMHDLQITCSEMQQKVELLRYESEKLQQENSILRNEITTLNEEDSISNLKLGTLNGSQE | |
| EMWQKTETVKQENAAVQKMVENLKKQISELKIKNQQLDLENTELSQKNSQNQEKLQELNQ | |
| RLTEMLCQKEKEPGNSALEEREQEKFNLKEELERCKVQSSTLVSSLEAELSEVKIQTHIV | |
| QQENHLLKDELEKMKQLHRCPDLSDFQQKISSVLSYNEKLLKEKEALSEELNSCVDKLAK | |
| SSLLEHRIATMKQEQKSWEHQSASLKSQLVASQEKVQNLEDTVQNVNLQMSRMKSDLRVT | |
| QQEKEALKQEVMSLHKQLQNAGGKSWAPEIATHPSGLHNQQKRLSWDKLDHLMNEEQQLL | |
| WQENERLQTMVQNTKAELTHSREKVRQLESNLLPKHQKHLNPSGTMNPTEQEKLSLKREC | |
| DQFQKEQSPANRKVSQMNSLEQELETIHLENEGLKKKQVKLDEQLMEMQHLRSTATPSPS | |
| PHAWDLQLLQQQACPMVPREQFLQLQRQLLQAERINQHLQEELENRTSETNTPQGNQEQL | |
| VTVMEERMIEVEQKLKLVKRLLQEKVNQLKEQLCKNTKADAMVKDLYVENAQLLKALEVT | |
| EQRQKTAEKKNYLLEEKIASLSNIVRNLTPAPLTSTPPLRS | |

### Examples 4-12

### Identification of Protein-protein Interactions

A yeast two-hybrid system as described in Example 1 using amino acids of the bait as set forth in Table 13 was performed. The clone that was identified by this procedure for each bait is set forth in Table 13 as the prey. The “AA” refers to the amino acids of the bait or prey. The “NUC” refers to the nucleotides of the bait or prey. The Accession numbers refer to GB: GenBank accession numbers.

TABLE 13

| Ex. | BAIT | ACCESSION | COORDINATES | PREY | ACCESSION | COORDINATES |
|---|---|---|---|---|---|---|
| 4 | LXR-alpha | GB: U22662 | AA: 95-156 | zyxin | GB: X94991 | AA 323-572 |
| 5 | LXR-alpha | GB: U22662 | AA 95-156 | LIMS1 | GB: U09284 | AA 25-90 |
| 6 | LXR-alpha | GB: U22662 | AA 95-277 | Homer-3 | GB: NM_0043838 | AA 182-354 |
| 7 | LXR-alpha | GB: U22662 | AA 95-277 | RACK1 | GB: M24194 | AA 178-317 |
| 8 | LXR-alpha | GB: U22662 | AA 257-448 | EIF3S1 | GB: NM_003758 | AA 94-254 |
| 9 | LXR-alpha | GB: U22662 | AA 156-448 | EIF3S1 | GB: NM_003758 | AA 94-254 |
| 10 | LXR-alpha | GB: U22662 | AA 95-277 | PSMD11 | GB: NM_002815 | AA 87-422 |
| 11 | LXR-alpha | GB: U22662 | AA 257-448 | KIAA0610 | GB: AB011182 | AA 36-245 |
| 12 | LXR-alpha | GB: U22662 | AA 156-448 | CIR | GB: U03644 | AA 226-450 |

### Example 13

### Generation of Polyclonal Antibody Against Protein Complexes

As shown above, LXR-alpha interacts with utrophin to form a complex. A complex of the two proteins is prepared, e.g., by mixing purified preparations of each of the two proteins. If desired, the protein complex can be stabilized by cross-linking the proteins in the complex, by methods known to those of skill in the art. The protein complex is used to immunize rabbits and mice using a procedure similar to that described by Harlow et al. (1988). This procedure has been shown to generate Abs against various other proteins (for example, see Kraemer et al., 1993).

Briefly, purified protein complex is used as immunogen in rabbits. Rabbits are immunized with 100 μg of the protein in complete Freund's adjuvant and boosted twice in three-week intervals, first with 100 μg of immunogen in incomplete Freund's adjuvant, and followed by 100 μg of immunogen in PBS. Antibody-containing serum is collected two weeks thereafter. The antisera is preadsorbed with LXR-alpha and utrophin, such that the remaining antisera comprises antibodies which bind conformational epitopes, i.e., complex-specific epitopes, present on the LXR-alpha-utrophin complex but not on the monomers.

Polyclonal antibodies against each of the complexes set forth in Tables 1-10 are prepared in a similar manner by mixing the specified proteins together, immunizing an animal and isolating antibodies specific for the protein complex, but not for the individual proteins.

Polyclonal antibodies against the protein set forth in Table 12 are prepared in a similar manner by immunizing an animal with the protein and isolating antibodies specific for the protein.

### Example 14

### Generation of Monoclonal Antibodies Specific for Protein Complexes

Monoclonal antibodies are generated according to the following protocol. Mice are immunized with immunogen comprising LXR-alpha/utrophin complexes conjugated to keyhole limpet hemocyanin using glutaraldehyde or EDC as is well known in the art. The complexes can be prepared as described in Example 13, and may also be stabilized by cross-linking. The immunogen is mixed with an adjuvant. Each mouse receives four injections of 10 to 100 μg of immunogen, and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single-cell suspension is prepared (Harlow et al., 1988). Cell fusions are performed essentially as described by Kohler et al. (1975). Briefly, P3.65.3 myeloma cells (American Type Culture Collection, Rockville, Md.) or NS-1 myeloma cells are fused with immune spleen cells using polyethylene glycol as described by Harlow et al. (1988). Cells are plated at a density of 2×10⁵ cells/well in 96-well tissue culture plates. Individual wells are examined for growth, and the supernatants of wells with growth are tested for the presence of LXR-alpha/utrophin complex-specific antibodies by ELISA or RIA using LXR-alpha/utrophin complex as target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to LXR-alpha alone or to utrophin alone, to determine which are specific for the LXR-alpha/utrophin complex as opposed to those that bind to the individual proteins.

Monoclonal antibodies against each of the complexes set forth in Tables 1-10 are prepared in a similar manner by mixing the specified proteins together, immunizing an animal, fusing spleen cells with myeloma cells and isolating clones which produce antibodies specific for the protein complex, but not for the individual proteins.

Monoclonal antibodies against the protein set forth in Table 12 are prepared in a similar manner by immunizing an animal with the protein, fusing spleen cells with myeloma cells and isolating clones which produce antibodies specific for the protein.

### Example 15

### In Vitro Identification of Modulators for Protein-protein Interactions

The present invention is useful in screening for agents that modulate the interaction of LXR-alpha and utrophin. The knowledge that LXR-alpha and utrophin form a complex is useful in designing such assays. Candidate agents are screened by mixing LXR-alpha and utrophin (a) in the presence of a candidate agent, and (b) in the absence of the candidate agent. The amount of complex formed is measured for each sample. An agent modulates the interaction of LXR-alpha and utrophin if the amount of complex formed in the presence of the agent is greater than (promoting the interaction), or less than (inhibiting the interaction) the amount of complex formed in the absence of the agent. The amount of complex is measured by a binding assay, which shows the formation of the complex, or by using antibodies immunoreactive to the complex.

Briefly, a binding assay is performed in which immobilized LXR-alpha is used to bind labeled utrophin. The labeled utrophin is contacted with the immobilized LXR-alpha under aqueous conditions that permit specific binding of the two proteins to form a LXR-alpha/utrophin complex in the absence of an added test agent. Particular aqueous conditions may be selected according to conventional methods. Any reaction condition can be used as long as specific binding of LXR-alpha/utrophin occurs in the control reaction. A parallel binding assay is performed in which the test agent is added to the reaction mixture. The amount of labeled utrophin bound to the immobilized LXR-alpha is determined for the reactions in the absence or presence of the test agent. If the amount of bound, labeled utrophin in the presence of the test agent is different than the amount of bound labeled utrophin in the absence of the test agent, the test agent is a modulator of the interaction of LXR-alpha and utrophin.

Candidate agents for modulating the interaction of each of the protein complexes set forth in Tables 1-10 are screened in vitro in a similar manner.

### Example 16

### In Vivo Identification of Modulators for Protein-protein Interactions

In addition to the in vitro method described in Example 15, an in vivo assay can also be used to screen for agents which modulate the interaction of LXR-alpha and utrophin. Briefly, a yeast two-hybrid system is used in which the yeast cells express (1) a first fusion protein comprising LXR-alpha or a fragment thereof and a first transcriptional regulatory protein sequence, e.g., GAL4 activation domain, (2) a second fusion protein comprising utrophin or a fragment thereof and a second transcriptional regulatory protein sequence, e.g., GAL4 DNA-binding domain, and (3) a reporter gene, e.g., β-galactosidase, which is transcribed when an intermolecular complex comprising the first fusion protein and the second fusion protein is formed. Parallel reactions are performed in the absence of a test agent as the control and in the presence of the test agent. A functional LXR-alpha/utrophin complex is detected by detecting the amount of reporter gene expressed. If the amount of reporter gene expression in the presence of the test agent is different than the amount of reporter gene expression in the absence of the test agent, the test agent is a modulator of the interaction of LXR-alpha and utrophin.

Candidate agents for modulating the interaction of each of the protein complexes set forth in Tables 1-10 are screened in vivo in a similar manner.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### Bibliography

Aronheim et al., 1997. Isolation of an AP-1 repressor by a novel method for detecting protein-protein interactions. Mol. Cell. Biol. 17:3094-3102.

Altschul, S. F. et al. (1990). Basic local alignment search tool. J. Mol. Biol. 215:403-410.

Altschul, S. F. et al. (1997). Gapped BLAST and PSI-BLAST: a new generation of protein database search programs. Nucl. Acids Res. 25:3389-3402.

Banres P. J. (1998). Anti-inflammatory actions of glucocorticoids: molecular mechanisms. Clinical Science 94:557-572.

Bartel, P. L. et al. (1993). “Using the 2-hybrid system to detect protein-protein interactions.” In: Cellular Interactions in Development: A Practical Approach, Oxford University Press, pp. 153-179.

Bartel, P. L. et al. (1996). A protein linkage map of Escherichia coli bacteriophage T7. Nat Genet 12:72-77.

Bartel, P. L. and Fields, S. (1997). The Yeast Two-Hybrid System. New York: Oxford University Press.

Brown, A. J. and Jessup, W. (1999). Oxysterols and atherosclerosis. Atherosclerosis 142:1-28.

Chevray, P. M. and Nathans, D. N. (1992). Protein interaction cloning in yeast: identification of mammalian proteins that interact with the leucine zipper of Jun. Proc. Natl. Acad. Sci. USA 89:5789-5793.

DeFranco, D. et al. (1998). Molecular chaperones and subcellular trafficking of steroid receptors. J. Steroid Biochem. and Mol. Biol. 65:51-58.

Duprez, D. et al. (2000). Aldosterone and vascular damage. Curr. Hypertens. Rep. 2:327-34.

Fields S and Song O-K (1989). A novel genetic system to detect protein-protein interactions. Nature 340:245-246.

Geller, D. S. et al. (2000). Activating mineralocorticoid receptor mutation in hypertension exacerbated by pregnancy. Science 289:119-123.

Harlow et al. (1988). Antibodies: A Laboratory Manual (Cold Spring Harbor Laboratory, Cold Spring Harbor, N.Y.).

Haynes, M. P. et al. (2000). Molecular mechanisms of estrogen actions ion the vasculature. J. Nucl. Cardiol. 7:500-508.

Hong, Y. R. et al. (2000). Cloning and characterization of a novel human ninein protein that interacts with the glycogen synthase kinase 3beta. Biochim Biophys Acta 1492:513-6.

Hsieh, J. J. et al. (1999). CIR, a corepressor linking the DNA binding factor CBF1 to the histone deacetylase complex. Proc Natl Acad Sci USA. 96:23-8.

Klein-Hitpass, L. et al. (1998). Targets of activated steroid hormone receptors: basal transcription factors and receptor interacting proteins. J. Mol. Med. 76:490-496.

Kohler, G. and Milstein, C. (1975). Continuous cultures of fused cells secreting antibody of predefined specificity. Nature 256:495-497.

Kraemer, F. B. et al. (1993). Detection of hormone-sensitive lipase in various tissues. I. Expression of an HSL/bacterial fusion protein and generation of anti-HSL antibodies. J. Lipid Res. 34:663-672.

Leanna, C. A. and Hannink, M. (1996). The reverse two-hybrid system: a genetic scheme for selection against specific protein/protein interactions. Nucl. Acids Res. 24:3341-3347.

Mack, G. J. et al. (1998). Autoantibodies to a group of centrosomal proteins in human autoimmune sera reactive with the centrosome. Arthritis Rheum. 41:551-8.

Mangelsdorf, D. et al. (1995). The nuclear receptor superfamily: the second decade. Cell 83:835-839.

Moras, D. and Gronemeyer, H. (1998). The nuclear receptor ligand-binding domain: structure and function. Curr. Op. Cell Biol. 10:384-391.

Schroepfer, G. J. (2000). Oxysterols: modulators of cholesterol metabolism and other processes. Physiol. Rev. 80:361-554.

Tu, Y. et al. (1998). Nck-2, a novel Src homology2/3-containing adaptor protein that interacts with the LIM-only protein PINCH and components of growth factor receptor kinase-signaling pathways. Mol Biol Cell. 9:3367-82.

Wetmur, J. G. and Davidson, N. (1968). “Kinetics of renaturation of DNA.” J. Mol. Biol. 31:349-370.

Whitfield, G. et al. (1999). Steroid hormone receptors: evolution, ligands, and molecular basis of biologic function. J. Biol. Chem. Suppl 32-33:110-122.

Wolffe, A. et al. (1997). Chromatin remodeling by steroid and nuclear receptors. Cell Research 7:127-142.

PCT Published Application No. WO 97/27296

PCT Published Application No. WO 99/65939

U.S. Pat. No. 5,622,852

U.S. Pat. No. 5,773,218

What is claimed is:

1. An isolated protein complex comprising two proteins, the protein complex selected from the group consisting of:
(i) a complex of a first protein and a second protein;
(ii) a complex of a fragment of said first protein and said second protein;
(iii) a complex of said first protein and a fragment of said second protein; and
(iv) a complex of a fragment of said first protein and a fragment of said second protein, wherein said first protein is LXR-alpha and said second protein is selected from the group consisting of utrophin, zyxin, LIMS1, PN7771, Homer-3, RACK1, EIF3S1, PSMD11, KIAA0610 and CIR.

2. The protein complex of claim 1, wherein said protein complex comprises said first protein and said second protein.

3. The protein complex of claim 1, wherein said protein complex comprises a fragment of said first protein and said second protein or said first protein and a fragment of said second protein.

4. The protein complex of claim 1, wherein said protein complex comprises fragments of said first protein and said second protein.

5. An isolated antibody selectively immunoreactive with a protein complex of claim 1.

6. The antibody of claim 5, wherein said antibody is a monoclonal antibody.

7. A method for diagnosing a physiological disorder in an animal, which comprises assaying for:
(a) whether a protein complex set forth in claim 1 is present in a tissue extract;
(b) the ability of proteins to form a protein complex set forth in claim 1; and
(c) a mutation in a gene encoding a protein of a protein complex set forth in claim 1.

8. The method of claim 7, wherein said animal is a human.

9. The method of claim 8, wherein said physiological disorder is selected from the group consisting of disorders associated with cholesterol homeostasis and atherogenesis.

10. The method of claim 7, wherein the diagnosis is for a predisposition to said physiological disorder.

11. The method of claim 7, wherein the diagnosis is for the existence of said physiological disorder.

12. The method of claim 7, wherein said physiological disorder is selected from the group consisting of disorders associated with cholesterol homeostasis and atherogenesis.

13. The method of claim 7, wherein said assay comprises a yeast two-hybrid assay.

14. The method of claim 7, wherein said assay comprises measuring in vitro a complex formed by combining the proteins of the protein complex, said proteins isolated from said animal.

15. The method of claim 14, wherein said complex is measured by binding with an antibody specific for said complex.

16. The method of claim 7, wherein said assay comprises mixing an antibody specific for said protein complex with a tissue extract from said animal and measuring the binding of said antibody.

17. A method for determining whether a mutation in a gene encoding one of the proteins of a protein complex set forth in claim 1 is useful for diagnosing a physiological disorder, which comprises assaying for the ability of said protein with said mutation to form a complex with the other protein of said protein complex, wherein an inability to form said complex is indicative of said mutation being useful for diagnosing a physiological disorder.

18. The method of claim 17, wherein said gene is an animal gene.

19. The method of claim 18, wherein said animal is a human.

20. The method of claim 19, wherein said physiological disorder is selected from the group consisting of disorders associated with cholesterol homeostasis and atherogenesis.

21. The method of claim 17, wherein the diagnosis is for a predisposition to a physiological disorder.

22. The method of claim 17, wherein the diagnosis is for the existence of a physiological disorder.

23. The method of claim 17, wherein said assay comprises a yeast two-hybrid assay.

24. The method of claim 17, wherein said assay comprises measuring in vitro a complex formed by combining the proteins of the protein complex, said proteins isolated from an animal.

25. The method of claim 24, wherein said animal is a human.

26. The method of claim 24, wherein said complex is measured by binding with an antibody specific for said complex.

27. A non-human animal model for a physiological disorder wherein the genome of said animal or an ancestor thereof has been modified such that the formation of a protein complex set forth in claim 1 has been altered.

28. The non-human animal model of claim 27, wherein said physiological disorder is selected from the group consisting of disorders associated with cholesterol homeostasis and atherogenesis.

29. The non-human animal model of claim 27, wherein the formation of said protein complex has been altered as a result of:
(a) over-expression of at least one of the proteins of said protein complex;
(b) replacement of a gene for at least one of the proteins of said protein complex with a gene from a second animal and expression of said protein;
(c) expression of a mutant form of at least one of the proteins of said protein complex;
(d) a lack of expression of at least one of the proteins of said protein complex; or
(e) reduced expression of at least one of the proteins of said protein complex.

30. A cell line obtained from the animal model of claim 27.

31. A non-human animal model for a physiological disorder, wherein the biological activity of a protein complex set forth in claim 1 has been altered.

32. The non-human animal model of claim 31, wherein said physiological disorder is selected from the group consisting of disorders associated with cholesterol homeostasis and atherogenesis.

33. The non-human animal model of claim 31, wherein said biological activity has been altered as a result of:
(a) disrupting the formation of said complex; or
(b) disrupting the action of said complex.

34. The non-human animal model of claim 31, wherein the formation of said complex is disrupted by binding an antibody to at least one of the proteins which form said protein complex.

35. The non-human animal model of claim 31, wherein the action of said complex is disrupted by binding an antibody to said complex.

36. The non-human animal model of claim 31, wherein the formation of said complex is disrupted by binding a small molecule to at least one of the proteins which form said protein complex.

37. The non-human animal model of claim 31, wherein the action of said complex is disrupted by binding a small molecule to said complex.

38. A cell in which the genome of cells of said cell line has been modified to produce at least one protein complex set forth in claim 1.

39. A cell line in which the genome of the cells of said cell line has been modified to eliminate at least one protein of a protein complex set forth in claim 1.

40. A composition comprising:
a first expression vector having a nucleic acid encoding a first protein or a homologue or derivative or fragment thereof; and
a second expression vector having a nucleic acid encoding a second protein, or a homologue or derivative or fragment thereof, wherein said first and said second proteins are the proteins of claim 1.

41. A host cell comprising:
a first expression vector having a nucleic acid encoding a first protein which is first protein or a homologue or derivative or fragment thereof; and
a second expression vector having a nucleic acid encoding a second protein which is second protein, or a homologue or derivative or fragment thereof thereof, wherein said first and said second proteins are the proteins of claim 1.

42. The host cell of claim 41, wherein said host cell is a yeast cell.

43. The host cell of claim 41, wherein said first and second proteins are expressed in fusion proteins.

44. The host cell of claim 41, wherein one of said first and second nucleic acids is linked to a nucleic acid encoding a DNA binding domain, and the other of said first and second nucleic acids is linked to a nucleic acid encoding a transcription-activation domain, whereby two fusion proteins can be produced in said host cell.

45. The host cell of claim 41, further comprising a reporter gene, wherein the expression of the reporter gene is determined by the interaction between the first protein and the second protein.

46. A method for screening for drug candidates capable of modulating the interaction of the proteins of a protein complex, the protein complex selected from the group consisting of the protein complexes of claim 1, said method comprising
(i) combining the proteins of said protein complex in the presence of a drug to form a first complex;
(ii) combining the proteins in the absence of said drug to form a second complex;
(iii) measuring the amount of said first complex and said second complex; and
(iv) comparing the amount of said first complex with the amount of said second complex,
Wherein if the amount of said first complex is greater than, or less than the amount of said second complex, then the drug is a drug candidate for modulating the interaction of the proteins of said protein complex.

47. The method of claim 46, wherein said screening is an in vitro screening.

48. The method of claim 46, wherein said complex is measured by binding with an antibody specific for said protein complexes.

49. The method of claim 46, wherein if the amount of said first complex is greater than the amount of said second complex, then said drug is a drug candidate for promoting the interaction of said proteins.

50. The method of claim 46, wherein if the amount of said first complex is less than the amount of said second complex, then said drug is a drug candidate for inhibiting the interaction of said proteins.

51. A drug useful for treating a physiological disorder identified by the method of claim 46.

52. The drug of claim 51, wherein said physiological disorder is selected from the group consisting of disorders associated with cholesterol homeostasis and atherogenesis.

53. A method of screening for drug candidates useful in treating a physiological disorder which comprises the steps of:
(a) measuring the activity of a protein selected from the goup consisting of a first protein and a second protein in the presence of a drug, wherein said first and second proteins are selected from the group consisting of the proteins of claim 1,
(b) measuring the activity of said protein in the absence of said drug, and
(c) comparing the activity measured in steps (1) and (2),
Wherein if there is a difference in activity, then said drug is a drug candidate for treating said physiological disorder.

54. A drug useful for treating a physiological disorder identified by the method of claim 53.

55. The drug of claim 54, wherein said physiological disorder is selected from the group consisting of disorders associated with cholesterol homeostasis and atherogenesis.

56. A method for selecting modulators of a protein complex formed between a first protein or a homologue or derivative or fragment thereof and a second protein or a homologue or derivative or fragment thereof, wherein said first and second proteins are selected from the group consisting of the proteins of claim 1, said method comprising:
providing the protein complex;
contacting said protein complex with a test compound; and
determining the presence or absence of binding of said test compound to said protein complex.

57. A modulator useful for treating a physiological disorder identified by the method of claim 56.

58. The modulator of claim 57, wherein said physiological disorder is selected from the group consisting of disorders associated with cholesterol homeostasis and atherogenesis.

59. A method for selecting modulators of an interaction between a first protein and a second protein, said first protein or a homologue or derivative or fragment thereof and said second protein or a homologue or derivative or fragment thereof, wherein said first and second proteins are selected from the group consisting of the proteins of claim 1, said method comprising:
contacting said first protein with said second protein in the presence of a test compound; and
determining the interaction between said first protein and said second protein.

60. The method of claim 59, wherein at least one of said first and second proteins is a fusion protein having a detectable tag.

61. The method of claim 59, wherein said step of determining the interaction between said first protein and said second protein is conducted in a substantially cell free environment.

62. The method of claim 59, wherein the interaction between said first protein and said second protein is determined in a host cell.

63. The method of claim 62, wherein said host cell is a yeast cell.

64. The method of claim 59, wherein said test compound is provided in a phage display library.

65. The method of claim 59, wherein said test compound is provided in a combinatorial library.

66. A modulator useful for treating a physiological disorder identified by the method of claim 59.

67. The modulator of claim 66, wherein said physiological disorder is selected from the group consisting of disorders associated with cholesterol homeostasis and atherogenesis.

68. A method for selecting modulators of a protein complex formed from a first protein or a homologue or derivative or fragment thereof, and a second protein or a homologue or derivative or fragment thereof, wherein said first and second proteins are selected from the group consisting of the proteins of claim 1, said method comprising:
contacting said protein complex with a test compound; and
determining the interaction between said first protein and said second protein.

69. A modulator useful for treating a physiological disorder identified by the method of claim 68.

70. The modulator of claim 69, wherein said physiological disorder is selected from the group consisting of disorders associated with cholesterol homeostasis and atherogenesis.

71. A method for selecting modulators of an interaction between a first polypeptide and a second polypeptide, said first polypeptide being a first protein or a homologue or derivative or fragment thereof and said second polypeptide being a second protein or a homologue or derivative or fragment thereof, wherein said first and second proteins are selected from the group consisting of the proteins of claim 1, said method comprising:
providing in a host cell a first fusion protein having said first polypeptide, and a second fusion protein having said second polypeptide, wherein a DNA binding domain is fused to one of said first and second polypeptides while a transcription-activating domain is fused to the other of said first and second polypeptides;
providing in said host cell a reporter gene, wherein the transcription of the reporter gene is determined by the interaction between the first polypeptide and the second polypeptide;
allowing said first and second fusion proteins to interact with each other within said host cell in the presence of a test compound; and
determining the presence or absence of expression of said reporter gene.

72. The method of claim 71, wherein said host cell is a yeast cell.

73. A modulator useful for treating a physiological disorder identified by the method of claim 71.

74. The modulator of claim 73, wherein said physiological disorder is selected from the group consisting disorders associated with cholesterol homeostasis and atherogenesis.

75. A method for identifying a compound that binds to a protein in vitro, wherein said protein is selected from the group consisting of the proteins of claim 1, said method comprising:
contacting a test compound with said protein for a time sufficient to form a complex and
detecting for the formation of a complex by detecting said protein or the compound in the complex, so that if a complex is detected, a compound that binds to protein is identified.

76. A compound useful for treating a physiological disorder identified by the method of claim 75.

77. The compound of claim 76, wherein said physiological disorder is selected from the group consisting of disorders associated with cholesterol homeostasis and atherogenesis.

78. A method for selecting modulators of an interaction between a first polypeptide and a second polypeptide, said first polypeptide being a first protein or a homologue or derivative or fragment thereof and said second polypeptide being a second protein or a homologue or derivative or fragment thereof, wherein said first and second proteins are selected from the group consisting of the proteins of claim 1, said method comprising:
providing atomic coordinates defining a three-dimensional structure of a protein complex formed by said first polypeptide and said second polypeptide; and
designing or selecting compounds capable of modulating the interaction between a first polypeptide and a second polypeptide based on said atomic coordinates.

79. A modulator useful for treating a physiological disorder identified by the method of claim 78.

80. The modulator of claim 79, wherein said physiological disorder is selected from the group consisting of disorders associated with cholesterol homeostasis and atherogenesis.

81. A method for providing inhibitors of an interaction between a first polypeptide and a second polypeptide, said first polypeptide being a first protein or a homologue or derivative or fragment thereof and said second polypeptide being a second protein or a homologue or derivative or fragment thereof, wherein said first and second proteins are selected from the group consisting of the proteins of claim 1, said method comprising:
providing atomic coordinates defining a three-dimensional structure of a protein complex formed by said first polypeptide and said second polypeptide; and
designing or selecting compounds capable of interfering with the interaction between a first polypeptide and a second polypeptide based on said atomic coordinates.

82. An inhibitor useful for treating a physiological disorder identified by the method of claim 81.

83. The inhibitor of claim 82, wherein said physiological disorder is selected from the group consisting of disorders associated with cholesterol homeostasis and atherogenesis.

84. A method for selecting modulators of a protein, wherein said protein is selected from the group consisting of the proteins of claim 1, said method comprising:
contacting said protein with a test compound; and
determining binding of said test compound to said protein.

85. The method of claim 84, wherein said test compound is provided in a phage display library.

86. The method of claim 84, wherein said test compound is provided in a combinatorial library.

87. A modulator useful for treating a physiological disorder identified by the method of claim 84.

88. The modulator of claim 87, wherein said physiological disorder is selected from the group consisting of disorders associated with cholesterol homeostasis and atherogenesis.

89. A method for modulating, in a cell, a protein complex having a first protein interacting with a second protein, wherein said first and second proteins are selected from the group consisting of the proteins of claim 1, said method comprising:
administering to said cell a compound capable of modulating said protein complex.

90. The method of claim 89, wherein said compound is selected from the group consisting of:
(a) a compound which is capable of interfering with the interaction between said first protein and said second protein,
(b) a compound which is capable of binding at least one of said first protein and said second protein,
(c) a compound which comprises a peptide having a contiguous span of amino acids of at least 4 amino acids of said second protein and capable of binding said first protein,
(d) a compound which comprises a peptide capable of binding said first protein and having an amino acid sequence of from 4 to 30 amino acids that is at least 75% identical to a contiguous span of amino acids of said second protein of the same length,
(e) a compound which comprises a peptide having a contiguous span of amino acids of at least 4 amino acids of said first protein and capable of binding said second protein,
(f) a compound which comprises a peptide capable of binding said second protein and having an amino acid sequence of from 4 to 30 amino acids that is at least 75% identical to a contiguous span of amino acids of said first protein of the same length,
(g) a compound which is an antibody immunoreactive with said first protein or said second protein,
(h) a compound which is a nucleic acid encoding an antibody immunoreactive with said first protein or said second protein,
(i) a compound which modulates the expression of said first protein or said second protein,
(j) a compound which is an antisense compound or a ribozyme specifically hybridizing to a nucleic acid encoding said first protein or complement thereof, and
(k) a compound which is an antisense compound or a ribozyme specifically hybridizing to a nucleic acid encoding said second protein or complement thereof.

91. A method for modulating, in a cell, a protein complex having a first protein interacting with a second protein, wherein said first and second proteins are selected from the group consisting of the proteins of claim 1, said method comprising:
administering to said cell a peptide capable of interfering with the interaction between said first protein and said second protein, wherein said peptide is associated with a transporter capable of increasing cellular uptake of said peptide.

92. The method of claim 91, wherein said peptide is covalently linked to said transporter which is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histine oligomers, D-histine oligomers, L-omithine oligomers, D-ornithine oligomers, short peptide sequences derived from fibroblast growth factor, Galparan, and HSV-1 structural protein VP22, and peptoid analogs thereof.

93. A method for modulating, in a cell, the interaction of a protein with a ligand, wherein said protein is selected from the group consisting of the first or second proteins of claim 1, said method comprising:
administering to said cell a compound capable of modulating said interaction.

94. The method of claim 93, wherein said protein is one of said first or second proteins and said ligand is the other of said first or second proteins.

95. The method of claim 93, wherein said compound is selected from the group consisting of:
(a) a compound which interferes with said interaction,
(b) a compound which binds to said protein or said ligand,
(c) a compound which comprises a peptide having a contiguous span of amino acids of at least 4 amino acids of said protein and capable of binding said ligand,
(d) a compound which comprises a peptide capable of binding said ligand and having an amino acid sequence of from 4 to 30 amino acids that is at least 75% identical to a contiguous span of amino acids of said protein of the same length,
(e) a compound which is an antibody immunoreactive with said protein or said ligand,
(f) a compound which is a nucleic acid encoding an antibody immunoreactive with said ligand or said protein,
(g) a compound which modulates the expression of said protein or said ligand, and
(h) a compound which is an antisense compound or a ribozyme specifically hybridizing to a nucleic acid encoding said ligand or said protein or complement thereof.

96. A method for modulating neuronal death in a patient having a physiological disorder comprising:
modulating a protein complex having a first protein interacting with a second protein, wherein said first and second proteins are selected from the group consisting of the proteins of claim 1.

97. The method of claim 96, wherein said physiological disorder is selected from the group consisting of disorders associated with cholesterol homeostasis and atherogenesis.

98. A method for modulating neuronal death in a patient having physiological disorder comprising:
administering to the patient a compound capable of modulating a protein complex having a first protein interacting with a second protein, wherein said first and second proteins are selected from the group consisting of the proteins of claim 1.

99. The method of claim 98, wherein said physiological disorder is selected from the group consisting of disorders associated with cholesterol homeostasis and atherogenesis.

100. The method of claim 98, wherein said compound is selected from the group consisting of:
(a) a compound which is capable of interfering with the interaction between said first protein and said second protein,
(b) a compound which is capable of binding at least one of said first protein and said second protein,
(c) a compound which comprises a peptide having a contiguous span of amino acids of at least 4 amino acids of a second protein and capable of binding a first protein,
(d) a compound which comprises a peptide capable of binding a first protein and having an amino acid sequence of from 4 to 30 amino acids that is at least 75% identical to a contiguous span of amino acids of a second protein of the same length,
(e) a compound which comprises a peptide having a contiguous span of amino acids of at least 4 amino acids of first protein and capable of binding a second protein,
(f) a compound which comprises a peptide capable of binding a second protein and having an amino acid sequence of from 4 to 30 amino acids that is at least 75% identical to a contiguous span of amino acids of a first protein of the same length,
(g) a compound which is an antibody immunoreactive with a first protein or a second protein,
(h) a compound which is a nucleic acid encoding an antibody immunoreactive with a first protein or a second protein,
(i) a compound which modulates the expression of a first protein or a second protein,
(j) a compound which is an antisense compound or a ribozyme specifically hybridizing to a nucleic acid encoding a first protein or complement thereof, and
(j) a compound which is an antisense compound or a ribozyme specifically hybridizing to a nucleic acid encoding a second protein or complement thereof.

101. A method for modulating neuronal death in a patient having physiological disorder comprising:
administering to said cell a peptide capable of interfering with the interaction between a first protein and a second protein, wherein said first and second proteins are selected from the group consisting of the proteins of claim 1, wherein said peptide is associated with a transporter capable of increasing cellular uptake of said peptide.

102. The method of claim 101, wherein said peptide is covalently linked to said transporter which is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histine oligomers, D-histine oligomers, L-ornithine oligomers, D-ornithine oligomers, short peptide sequences derived from fibroblast growth factor, Galparan, and HSV-1 structural protein VP22, and peptoid analogs thereof.

103. A method for treating a physiological disorder comprising:
administering to a patient in need of treatment a compound capable of modulating a protein complex having a first protein interacting with a second protein, wherein said first and second proteins are selected from the group consisting of the proteins of claim 1.

104. The method of claim 103, wherein said physiological disorder is selected from the group consisting of disorders associated with cholesterol homeostasis and atherogenesis.

105. The method of claim 103, wherein said compound is selected from the group consisting of:
(a) a compound which is capable of interfering with the interaction between said first protein and said second protein,
(b) a compound which is capable of binding at least one of said first protein and said second protein,
(c) a compound which comprises a peptide having a contiguous span of amino acids of at least 4 amino acids of said second protein and capable of binding said first protein,
(d) a compound which comprises a peptide capable of binding said first protein and having an amino acid sequence of from 4 to 30 amino acids that is at least 75% identical to a contiguous span of amino acids of said second protein of the same length,
(e) a compound which comprises a peptide having a contiguous span of amino acids of at least 4 amino acids of first protein and capable of binding said second protein,
(f) a compound which comprises a peptide capable of binding said second protein and having an amino acid sequence of from 4 to 30 amino acids that is at least 75% identical to a contiguous span of amino acids of said first protein of the same length,
(g) a compound which is an antibody immunoreactive with siad first protein or said second protein,
(h) a compound which is a nucleic acid encoding an antibody immunoreactive with said first protein or said second protein,
(i) a compound which modulates the expression of said first protein or said second protein,
(j) a compound which is an antisense compound or a ribozyme specifically hybridizing to a nucleic acid encoding a first protein or complement thereof,
(k) a compound which is an antisense compound or a ribozyme specifically hybridizing to a nucleic acid encoding a second protein or complement thereof, and
(l) a compound which is capable of strengthening the interaction between said first protein and said second protein.

106. A method for treating a physiological disorder comprising:
administering to said cell a peptide capable of interfering with the interaction between a first protein and a second protein, wherein said first and second proteins are selected from the group consisting of the proteins of claim 1, wherein said peptide is associated with a transporter capable of increasing cellular uptake of said peptide.

107. The method of claim 106, wherein said peptide is covalently linked to said transporter which is selected from the group consisting of penetrating, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histine oligomers, D-histine oligomers, L-ornithine oligomers, D-ornithine oligomers, short peptide sequences derived from fibroblast growth factor, Galparan, and HSV-1 structural protein VP22, and peptoid analogs thereof.

108. The method of claim 106, wherein said physiological disorder is selected from the group consisting of disorders associated with cholesterol homeostasis and atherogenesis.

109. A method for treating a physiological disorder comprising:
administering to a patient in need of treatment a compound capable of modulating the activity of a first protein or a second protein, wherein said first and second proteins are selected from the group consisting of the proteins of claim 1.

110. The method of claim 109, wherein said physiological disorder is selected from the group consisting of disorders associated with cholesterol homeostasis and atherogenesis.

111. The method of claim 109, wherein the activity is the interaction of said first protein or said second protein with a ligand.

112. The method of claim 111, wherein said ligand is the other of said first or second protein.

113. A method of modulating activity in a cell of a protein, said protein being first protein or a second protein selected from the group consisting of the proteins of claim 1, said method comprising:
administering to said cell a compound capable of modulating said protein.

114. The method of claim 113, wherein said compound is selected from the group consisting of:
(a) a compound which is capable of binding said protein,
(b) a compound which comprises a peptide having a contiguous span of at least 4 amino acids of a first protein and capable of binding a second protein,
(c) a compound which comprises a peptide capable of binding a second protein and having an amino acid sequence of from 4 to 30 amino acids that is at least 75% identical to a contiguous span of amino acids of a first protein of the same length,
(d) a compound which is an antibody immunoreactive with said protein,
(e) a compound which is a nucleic acid encoding an antibody immunoreactive with said protein, and
(f) a compound which is an antisense compound or a ribozyme specifically hybridizing to a nucleic acid encoding said protein or complement thereof.

115. A method for modulating activities of a protein in a cell, said protein being a first protein or a second protein selected from the group consisting of the proteins of claim 1, said method comprising:
administering to said cell a peptide having a contiguous span of at least 4 amino acids of one of said first or second proteins and capable of binding the other of said first or second proteins, wherein said peptide is associated with a transporter capable of increasing cellular uptake of said peptide.

116. The method of claim 115, wherein said peptide is covalently linked to said transporter which is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histine oligomers, D-histine oligomers, L-ornithine oligomers, D-ornithine oligomers, short peptide sequences derived from fibroblast growth factor, Galparan, and HSV-1 structural protein VP22, and peptoid analogs thereof.

117. An isolated nucleic acid encoding a protein comprising an amino acid sequence set forth in SEQ ID NO: 4.

118. The isolated nucleic acid sequence of claim 117 which comprises nucleotides 544-6960 of SEQ ID NO: 3 or complement thereof.

119. An isolated nucleic acid encoding a protein comprising an amino acid sequence which is at least 70% identical to the amino acid sequence set forth in SEQ ID NO: 4 and which is capable of interacting with LXR-alpha.

120. An isolated nucleic acid comprising a nucleotide sequence which is at least 60% identical to nucleotides 544-6960 of SEQ ID NO: 3 or complement thereof.

121. An isolated nucleic acid sequence comprising a nucleotide sequence set forth in SEQ ID NO: 3 or complement thereof.

122. An isolated nucleic acid comprising a contiguous span of at least 17 nucleotides of the nucleotide sequence set forth in SEQ ID NO: 3 or complement thereof.

123. The isolated nucleic acid of claim 122 comprising at least 21 nucleotides.

124. The isolated nucleic acid of claim 122 comprising at least 25 nucleotides.

125. The isolated nucleic acid of claim 122 comprising at least 30 nucleotides.

126. The isolated nucleic acid of claim 122 comprising at least 50 nucleotides.

127. An isolated nucleic acid comprising at least 21 nucleotides that encodes a contiguous span of at least 7 amino acids of the amino acid sequence set forth in SEQ ID NO: 4.

128. The isolated nucleic acid of claim 127 encoding at least 8 contiguous amino acids.

129. The isolated nucleic acid of claim 127 encoding at least 9 contiguous amino acids.

130. The isolated nucleic acid of claim 127 encoding at least 10 contiguous amino acids.

131. The isolated nucleic acid of claim 127 encoding at least 15 contiguous amino acids.

132. The isolated nucleic acid of claim 127 encoding at least 20 contiguous amino acids.

133. The isolated nucleic acid of claim 127 encoding at least 25 contiguous amino acids.

134. A nucleic acid vector comprising the isolated nucleic acid of claim 17.

135. A nucleic acid vector comprising the isolated nucleic acid of claim 18.

136. A nucleic acid vector comprising the isolated nucleic acid of claim 119.

137. A nucleic acid vector comprising the isolated nucleic acid of claim 124.

138. A nucleic acid vector comprising the isolated nucleic acid of claim 130.

139. A host cell comprising the isolated nucleic acid of claim 117.

140. A host cell comprising the isolated nucleic acid of claim 118.

141. A host cell comprising the isolated nucleic acid of claim 119.

142. A host cell comprising the isolated nucleic acid of claim 116.

143. A host cell comprising the isolated nucleic acid of claim 130.

144. A microarray comprising the isolated nucleic acid of claim 130.

145. An isolated polypeptide comprising an amino acid sequence set forth in SEQ ID NO: 4.

146. An isolated polypeptide comprising an amino acid sequence that is at least 70% identical to the amino acid sequence set forth in SEQ ID NO: 4 and capable of interacting with LXR-alpha.

147. An isolated polypeptide comprising a contiguous span of at least 8 amino acids of the amino acid sequence set forth in SEQ ID NO: 4.

148. The isolated polypeptide of claim 147 comprising a contiguous span of at least 10 amino acids.

149. The isolated polypeptide of claim 147 comprising a contiguous span of at least 12 amino acids.

150. The isolated polypeptide of claim 147 comprising a contiguous span of at least 15 amino acids.

151. The isolated polypeptide of claim 147 comprising a contiguous span of at least 17 amino acids.

152. The isolated polypeptide of claim 147 comprising a contiguous span of at least 20 amino acids.

153. An isolated polypeptide comprising an amino acid sequence of from 4 to 30 amino acids that is at least 75% identical to a contiguous span of amino acids of the amino acid sequence set forth in SEQ ID NO: 4 of the same length, wherein said isolated polypeptide is capable of interacting with LXR-alpha.

154. The isolated polypeptide of claim 153, wherein said amino acid sequence comprises from 8 to 20 amino acids.

155. An antibody which is specifically immunoreactive with the isolated polypeptide of claim 145.

156. An antibody which is specifically immunoreactive with the isolated polypeptide of claim 147.

157. A protein microarray comprising the isolated polypeptide of claim 145.

158. A protein microarray comprising the isolated polypeptide of claim 147.

159. A protein microarray comprising the isolated polypeptide of claim 154.

160. A method for making an isolated polypeptide comprising an amino acid sequence set forth in SEQ ID NO: 4, comprising:
providing an expression vector comprising a nucleic acid encoding said amino acid sequence; and
introducing said expression vector into a host cell such that said host cell producing the isolated polypeptide.
